(19) United States
(12) Patent Application Publication
(10) Pub. No.: US 2024/0091220 A1
(43) Pub. Date: Mar. 21, 2024

(54) METHODS OF MODULATING NEURONAL AND OLIGODENDROCYTE SURVIVAL

(71) Applicants: NEW YORK UNIVERSITY, New York, NY (US); THE BOARD OF TRUSTEES OF THE LELAND STANFORD JUNIOR UNIVERSITY, Palo Alto, CA (US)

(72) Inventors: Shane LIDDELOW, New York, NY (US); Kevin GUTTENPLAN, Portland, OR (US); Ben BARRES (Deceased), Palo Alto, CA (US)

(21) Appl. No.: 18/280,126

(22) PCT Filed: Mar. 3, 2022
(86) PCT No.: PCT/US22/18744

§ 371 Date: Sep. 1, 2023

Related U.S. Application Data
(60) Provisional application No. 63/156,713, filed on Mar. 4, 2021.

(51) Int. Cl. A61K 31/496; A61K 31/192; A61K 31/201; A61K 31/436; C12N 15/113

(52) U.S. Cl. CPC A61K 31/496; A61K 31/192; A61K 31/201; A61K 31/436; C12N 15/1137; C12N 2310/141

(57) ABSTRACT

The present disclosure relates to methods of inhibiting reactive astrocyte mediated neuronal and/or oligodendrocyte cell death in a subject. In one embodiment, the method involves administering an inhibitor of Elongation of Very Long Chain Fatty Acids Protein 1 (ELOVL1) to a subject having or at risk of having a condition mediated by reactive astrocytes, where the ELOVL1 inhibitor is administered in an amount effective to inhibit reactive astrocyte mediated neuronal and/or oligodendrocyte cell death in the subject. In another embodiment, the method involves administering an inhibitor of lipoapoptosis to a subject having or at risk of having a condition mediated by reactive astrocytes, where the inhibitor of lipoapoptosis is administered in an amount effective to inhibit reactive astrocyte mediate neuronal and/or oligodendrocyte cell death in the subject.

This application claims the priority benefit of U.S. Provisional Patent Application Ser. No. 63/156,713, filed Mar. 4, 2021, which is hereby incorporated by reference in its entirety.

### FIELD

The present invention relates to methods of inhibiting reactive astrocyte mediated neuronal and/or oligodendrocyte cell death in a subject.

### BACKGROUND

Astrocytes undergo functional changes in response to disease and injury of the central nervous system (CNS), but the mechanisms underlying these changes and their therapeutic relevance remain unclear (Liddelow and Barres, “Reactive Astrocytes: Production, Function, and Therapeutic Potential,” Immunity 46(6):957-967 (2017). In studies characterizing astrocytes following systemic inflammation, it was found that microglial-derived interleukin 1 alpha (IL-1α), tumor necrosis factor (TNF), and complement component 1q (C1q) are necessary and sufficient to induce astrocyte reactivity in neuroinflammatory contexts (Liddelow et al., “Neurotoxic Reactive Astrocytes are Induced by Activated Microglia,” Nature 541(7638):481-487 (2017)) Astrocytes activated by these cytokines in vitro secrete factors that are toxic to neurons and oligodendrocytes, but not to other CNS cells (Liddelow et al., “Neurotoxic Reactive Astrocytes are Induced by Activated Microglia,” Nature 541(7638):481-487 (2017)). In vivo, genomic deletion of Il1a, Tnf, and C1qa prevented death of retinal ganglion cell (RGC) neurons after optic nerve crush (ONC) or in the bead occlusion model of glaucoma (Guttenplan et al., “Neurotoxic Reactive Astrocytes Drive Neuronal Death after Retinal Injury,” Cell Rep. 31(12):107776 (2020)) and extended the lifespan of SODI^{G93A} ALS model mice (Guttenplan et al., “Knockout of Reactive Astrocyte Activating Factors Slows Disease Progression in an ALS Mouse Model,” Nat. Commun. 11(1):3753 (2020)). These results indicate that astrocytes may contribute to neurodegeneration, but the molecular agents that promote that neurodegeneration remain unknown and, hence, how to modulate such neurodegeneration also remains unknown.

The present invention is directed to overcoming these and other deficiencies in the art.

### SUMMARY

The present disclosure relates to a method of inhibiting reactive astrocyte mediated neuronal and/or oligodendrocyte cell death in a subject. The method involves administering an inhibitor of Elongation of Very Long Chain Fatty Acids Protein 1 (ELOVL1) to a subject having or at risk of having a condition mediated by reactive astrocytes, where the ELOVL1 inhibitor is administered in an amount effective to inhibit reactive astrocyte mediated neuronal and/or oligodendrocyte cell death in the subject.

Another aspect of the present disclosure relates to a method of inhibiting reactive astrocyte mediated neuronal and/or oligodendrocyte cell death in a subject. The method involves administering an inhibitor of lipoapoptosis to a subject having or at risk of having a condition mediated by reactive astrocytes, where the lipoapoptosis inhibitor is administered in an amount effective to inhibit reactive astrocyte mediate neuronal and/or oligodendrocyte cell death in the subject.

Astrocytes are essential regulators of the central nervous system's response to disease and injury and have been hypothesized to actively kill neurons in neurodegenerative disease. As described herein, biochemical methods were utilized to identify saturated lipids contained in ApoE/ApoJ lipoparticles as components of astrocyte-mediated toxicity in vitro and in vivo. This was entirely unexpected, as it was hypothesized that the long-sought, astrocyte-derived toxic factor would be a protein. It was found that eliminating the formation of long-chain saturated lipids by cell-type-specific knockout of the synthesis enzyme Elovl1 reduces astrocyte-mediated toxicity in vitro as well as in an acute axonal injury model in vivo. These results identify an entirely new mechanism by which astrocytes kill cells in the CNS. As described herein this process can be targeted and inhibited by therapeutic agents as a means for treating and/or preventing neurodegenerative diseases and other conditions arising from reactive astrocyte mediated CNS cell death.

### BRIEF DESCRIPTION OF THE DRAWINGS

FIGS. 1A-1I demonstrate that proteins are upregulated in reactive astrocyte conditioned media. FIG. 1A shows phase-contrast images of oligodendrocytes treated with quiescent or reactive astrocyte conditioned media (ACM; scale bar=100 μm). FIG. 1B are graphs showing the quantification of oligodendrocyte survival. FIG. 1C is a schematic showing the proteomics pipeline. FIG. 1D is a graph showing proteins detected in quiescent versus reactive astrocytes (complete proteomics at http://gliaomics.com/). FIG. 1E is a plot showing proteins in quiescent versus reactive ACM (C3, SERPING1, CST3—reactivity markers; APOJ, APOE—lipoproteins, SPARC—classical secreted protein). FIG. 1F shows bar graphs showing the toxicity of fractions from biochemical purifications of reactive ACM. Arrowheads indicate fraction used for subsequent purification. FIG. 1G is a plot showing proteins detected in quiescent versus reactive ACM purified by the columns in FIG. 1F (FIG. 16). FIG. 1H shows graphs providing ELISA quantification of ApoE and ApoJ in quiescent versus reactive ACM. FIG. 1I is an exemplary HPLC trace (Control HPLC, Reactive HPLC, plotted against Absorbance at 280 nm—FIGS. 8A-8C) of protein abundance in size exclusion HPLC. Traces show ELISA quantification of ApoE (APOE conc.) and ApoJ (APOJ conc.) in fractions associated with astrocytic HDLs. For all: * P<0.05; data represented as mean±s.e.m.; see FIG. 15 for statistics and data reporting.

FIGS. 2A-2I show differentially regulated lipids in reactive astrocytes. FIG. 2A is a graph showing that antibody pulldown of ApoE and ApoJ (ApoE/J), but not IgG control, reduces reactive ACM toxicity. FIG. 2B is graph showing that ACM from reactive astrocytes isolated from WT, ApoE^{-/-}, ApoJ^{-/-}, and ApoE^{-/-} ApoJ^{-/-} mice are similarly toxic. FIG. 2C is a graph showing that toxic ACM stripped of lipids by a Lipidex 3000 column are not toxic. Reactive ACM lipids eluted from the Lipidex 3000 column, but not HEK conditioned media lipids, are toxic. FIG. 2D is a plot showing that membranes isolated from reactive astrocytes, but not quiescent astrocytes or HEK cells, are toxic. FIG. 2E is an exemplary image of oligodendrocytes incorporating reconstituted lipoparticles (rHDL) composed of recombinant ApoE/J, ACM lipids, and fluoro-phosphatidylcholine (scale bar=100 μm). FIG. 2F is a graph showing rHDL composed of reactive ACM lipids are more toxic than those containing quiescent ACM lipids. FIG. 2G is a schematic showing the pipeline for unbiased lipidomics and metabolomics. FIG. 2H is a plot showing reactive and control astrocytes and ACM separate in PCA space by their lipidome. FIG. 2I is a heatmap of differentially-regulated lipids shows saturated, long-chain phospholipids are upregulated in reactive astrocytes and saturated, long-chain FFAs are upregulated in reactive ACM. For all: * P<0.05; data represented as mean±s.e.m.; see FIG. 15 for statistics and data reporting.

FIGS. 3A-3E demonstrate the mechanism of cell death from reactive astrocyte conditioned media. FIG. 3A is a schematic showing the lipoapoptotic cell death pathway (adapted from Cunha et al., “Death Protein 5 and p53-Upregulated Modulator of Apoptosis Mediate the Endoplasmic Reticulum Stress-Mitochondrial Dialog Triggering Lipotoxic Rodent and Human β-cell Apoptosis,” Diabetes 61:2763-2775 (2012), which is hereby incorporated by reference in its entirety). FIG. 3B is a graph showing that oligodendrocytes treated with toxic ACM undergo lipoapoptosis. FIG. 3C shows western blots quantified in FIG. 3B. FIG. 3D show exemplary images of oligodendrocytes from PUMA^{-/-} or WT mice treated with toxic ACM demonstrates resistance of PUMA^{-/-} oligodendrocytes to astrocyte-mediated toxicity (scale bar=100 μm). FIG. 3E is a graph showing survival quantification of oligodendrocytes isolated from WT, CHOP^{-/-}, and PUMA^{-/-} mice. For all: * P<0.05; data represented as mean±s.e.m.; see FIG. 15 for statistics and data reporting.

FIGS. 4A-4G show that conditional knockout of long-chain saturated lipid synthesis gene Elovl1 reduces reactive astrocyte toxicity. FIG. 4A is a schematic showing the experimental design for Elovl1 cKO mice. FIG. 4B is a targeted lipidomics plot showing decrease in long-chain saturated lipids (square) in Elovl1 cKO versus WT reactive astrocytes. FIG. 4C shows exemplary images of oligodendrocytes treated with Elovl1 cKO versus WT ACM (Calcein AM—live cells, ethidium homodimer (Ethd)—dead cells; scale bar=100 μm). FIG. 4D is a graph showing quantification of oligodendrocyte survival shows decreased toxicity of Elvol1 cKO versus WT reactive ACM, including when concentrated 10×. FIG. 4E is a graph showing reactive ACM and reactive ACM lipid-bearing reconstituted lipoparticles are toxic to retinal ganglion cells (RGCs) in vitro. FIG. 4F shows exemplary images of RGCs (RBPMS) in WT and Elovl1 cKO retinas after optic nerve crush (ONC; scale bar=50 μm). FIG. 4G is a plot showing quantification of RBPMS⁺ RGC number in Elovl1 cKO versus WT retinas after ONC, which shows astrocyte Elovl1 cKO is neuroprotective. For all: * P<0.05; data represented as mean±s.e.m.; see FIG. 15 for statistics and data reporting.

FIGS. 5A-5D show principal components analysis of protein mass spectrometry data. FIG. 5A is graph showing a number of significant proteins and PCA variation based on number of replicates of protein mass spectrometry that were required to have a non-zero spectral count to be considered for analysis. 3660 total unique proteins detected in astrocytes and 183 total unique proteins detected in ACM. 4 of 10 (4×) was chosen for final analysis. FIG. 5B shows PCA plots of cellular and ACM protein mass spectrometry of all proteins detected in at least 4 of 10 astrocytes samples see shows clear separation of the proteome and secretome of reactive versus control astrocytes. FIG. 5C is a graph showing quantification of differentially regulated proteins in reactive astrocytes and ACM (FDR<0.1). FIG. 5D is a chart showing the 10 most upregulated and downregulated proteins in reactive versus control astrocytes (bold=known reactivity markers).

FIGS. 6A-6B demonstrates the toxicity testing of various candidate toxic proteins. FIG. 6A are graphs showing the results of an experiment in which oligodendrocytes were treated with various doses of candidate toxic proteins found in the proteomics analysis study or from previous literature but not found to be toxic in the present study culture conditions. FIG. 6B is a graph showing that reactive ACM, but not Lcn2, Lgals1, or complement component C3 family members, is toxic to retinal ganglion cell (RGC) neuron cultures. All data represents N=¾ independent samples from 2 separate primary cell isolations; presented as mean±SEM.

FIGS. 7A-7B show the enrichment of toxic factors. FIG. 7A is a schematic diagram of sequential toxic factor enrichment via various biochemical purification columns. FIG. 7B is a graph showing validation that sequentially enriched reactive ACM is more toxic than sequentially enriched control ACM. Data represents 3 independent samples from 3 separate primary cell isolations.

FIGS. 8A-8C shows astrocyte lipoparticle analysis. FIG. 8A shows exemplary control and reactive protein abundance traces for HPLC size exclusion column. FIG. 8B is a graph showing the results of an ELISA showing an increase in ApoJ concentration within fractions associated with astrocyte HDL (individual data points represent independent samples from a single primary cell isolation; presented as mean±SEM). FIG. 8C shows the results of an ELISA on concentrated control versus reactive HPLC fractions associated with HDL, which shows more ApoE in reactive versus control. Control and reactive HDL fractions were combined and concentrated to achieve sufficient signal for ApoE ELISA so only one sample for control versus reactive was analyzed.

FIGS. 9A-9B demonstrate reconstituted HDL incorporation into cells. FIG. 9A is a graph showing AFUs of ApoE and ApoJ versus time. FIG. 9B shows exemplary images of fluorescently labeled reconstituted HDL incorporation into oligodendrocyte, microglia, endothelial cells, oligodendrocyte precursor cells (OPCs), retinal ganglion cell neurons (RGCs), and astrocytes in vitro. Note that all cells incorporate reconstituted lipoparticles except for endothelial cells. Experiment performed on 2 separate primary cell isolations for each cell type; scale bar=100 μm.

FIGS. 10A-10D demonstrate astrocyte metabolomics and lipidomics. FIG. 10A shows that reactive versus control astrocytes and ACM are somewhat separable in PCA space based on their metabolome, but less so than by their lipidome (FIGS. 2A-2I). FIG. 10B is a graph showing the distribution of MRM transitions selected for screening lipids. A total of 1547 transitions (used to ID lipid species) were organized into 11 MRM-based mass spectrometry methods (for lipid classes). FIG. 10C is a graph showing the quantification of differentially regulated lipids and metabolites in reactive astrocytes and ACM (FDR<0.1). FIG. 10D shows scatterplots of differentially regulated lipids in reactive versus control astrocytes and ACM highlights the overall abundance of differentially regulated lipids.

FIGS. 11A-11C demonstrate that saturated free fatty acids and phosphatidylcholines are toxic to oligodendrocytes. FIG. 11A is an image showing that cultured oligodendrocytes (phase) incorporate fluorescent C16:0 FFAs (green) upon treatment (0.5 μM; scale bar=150 μm). FIG. 11B is a dose curve of oligodendrocyte survival following treatment with C16:0 and C18:0 saturated FFAs shows that saturated FFAs are toxic to oligodendrocytes with longer chain lengths leading to greater toxicity (curve fits performed using one-phase decay model). FIG. 11C is a graph showing that long-chain saturated phosphatidylcholines (20:0) are toxic to oligodendrocytes in a dose-dependent fashion. Data, including representative image in subpanel a, represents N=4 independent samples from 3 separate primary cell isolations; presented as mean±SEM.

FIGS. 12A-12C provide further analysis of the mechanism of toxic factor induced cell death. FIG. 12A is a graph showing the results of an experiment in which various doses of ethoxyquin in DMSO was added to oligodendrocytes with or without 30 μg/ml reactive ACM. Simple linear regression analysis on increasing doses of ethoxyquin without reactive ACM (Slope=−0.0000025, P value [slope≠0]=0.1932) and with reactive ACM (Slope=−0.000001788, P value [slope≠0]=0.4194) failed to show a significant relationship between ethoxyquin concentration and survival, indicating that the free radical scavenger did not impact cell survival when treated in isolation and did not impact the toxicity of reactive ACM. This data, in addition to the data that Ferrostatin-1 has no effect on astrocyte toxicity (Liddelow et al., “Neurotoxic Reactive Astrocytes are Induced by Activated Microglia,” Nature 541(7638):481-487 (2017), which is hereby incorporated by reference in its entirety), indicates that lipid peroxidation may not mediate the ACM toxicity. FIG. 12B is a graph showing that siRNAs potently knock down the lipoapoptosis sensitivity modulated genes Scd1 and Insig1 in oligodendrocytes in vitro. FIG. 12C is a graph showing that knockdown of SCD and INSIG1, which bidirectionally modulate sensitivity to lipoapoptosis, bidirectionally modulate sensitivity of oligodendrocytes to toxic ACM. Data represents n=3 independent samples from 2 separate primary cell isolations; presented as mean±SEM.

FIGS. 13A-13E shows Elovl1 cKO validation. FIG. 13A are images showing GFP expression from NuTrap mice crossed to Gfap-Cre line used in this study, which shows efficient recombination in Slc1a3⁺ astrocytes (as identified by RNAscope in situ hybridization) of the ganglion cell layer (GCL, identified by DAPI staining of nuclei; ONL, outer nuclear layer; OPL, outer plexiform layer; INL, inner nuclear layer; IPL, inner plexiform layer). FIG. 13B shows DNA gels following PCR amplification of Elovl1 and Gfap in the retina and optic nerve, which shows a decrease in Elovl1 expression relative to Gfap expression in the Elovl1 cKO mouse visual system (white numbers indicate molecular weight markers). FIG. 13C is a graph showing the quantification of the decrease in Elovl1 expression relative to Gfap expression in the Elovl1 cKO retina (top) and optic nerve (bottom) N=4 animals per group, bars represent s.e.m., two-tailed Student's t-test). FIG. 13D is a graph showing targeted lipidomics of Elovl1 cKO ACM, which shows dampened upregulation of the long-chain saturated lipids normally upregulated in WT reactive ACM (black line indicates equal upregulation; red dots indicate lipids less upregulated in Elovl1 cKO versus WT ACM; black dot indicates a lipid less upregulated in WT ACM versus Elovl1 cKO ACM). FIG. 13E shows the separation of Elovl1 cKO and WT cell and ACM lipidomes in PCA space.

FIG. 14 shows Elovl1 cKo versus WT ACM toxicity over time. Toxicity of oligodendrocytes in response to Elvol1 cKO versus wt control, reactive, and concentrated reactive ACM over 96 hours (data represents mean±SEM of 6 experimental replicates each from 3 independent samples from 3 separate primary cell isolations; presented as mean±SEM).

FIG. 15 shows statistics and data reporting for FIGS. 1A-1J, 2A-2G, 3B-3G, and 4B-4H.

FIG. 16 shows the results of ApoE and ApoJ pulldown.

### DETAILED DESCRIPTION

The present disclosure relates to methods of inhibiting reactive astrocyte mediated neuronal and/or oligodendrocyte cell death in a subject.

According to one approach, the method involves administering an inhibitor of Elongation of Very Long Chain Fatty Acids Protein 1 (ELOVL1) to a subject having or at risk of having a condition mediated by reactive astrocytes, where the ELOVL1 inhibitor is administered in an amount effective to inhibit reactive astrocyte mediated neuronal and/or oligodendrocyte cell death in the subject.

According to another approach, the method involves administering an inhibitor of lipoapoptosis to a subject having or at risk of having a condition mediated by reactive astrocytes, where the lipoapoptosis inhibitor is administered in an amount effective to inhibit reactive astrocyte mediate neuronal and/or oligodendrocyte cell death in the subject.

Astrocytes, also known as astroglia, are star-shaped glial cells in the brain and spinal cord. Astrocytes function in biochemical support of endothelial cells that form the blood vessel-brain barrier, provide nutrition to nerve tissues, maintain extracellular ionic balance, and repair the brain and spinal cord after traumatic injury. However, it is known that reactive astrocytes are modified astrocytes that are toxic to neurons and can secrete signals capable of killing neurons (Liddelow et al., “Neurotoxic Reactive Astrocytes are Induced by Activated Microglia,” Nature 541(7638):481-487 (2017), which is hereby incorporated by reference in its entirety).

Thus, as used herein, the term “reactive astrocyte” refers to an astrocyte that responds to an external stimuli like inflammation, injury, neurodegeneration, infection, ischemia, stroke, autoimmune reactions, neurodegenerative diseases, and the like. In some embodiments, reactive astrocytes are characterized in that they become neurotoxic upon activation of IL-1α and TNFα or IL-1α, TNFα, and C1q signaling. Reactive astrocytes can induce death of other astrocytes, oligodendrocytes, or neurons by inhibiting the regeneration of nerve cells or secreting toxic substances. Reactive astrocytes may be defined and/or identified based on gene expression, including e.g., based on the expression of one or more reactive astrocyte markers including but not limited to e.g., H2.T23, Serping1, H2.D1, Ggta1, Iigp1, Gbp2, Fbln5, Ugtla, Fkbp5, Psmb8, Srgn, Amigo2, C3, Clef 1, Tgm1, Ptx3, S100a10, Sphk1, Cd109, Ptgs2, Emp1, Slc10a6, Tm4sf1, B3gnt5 and Cd14. Reactive astrocytes generally express or overexpress (e.g., as compared to resting astrocytes) one or more ‘pan reactive’ genes (i.e., genes having expression associated with reactive astrocytes of various subgroups). Pan reactive genes include but are not limited to e.g., Lcn2, Steap4, S1 pr3, Timpl, Hspbl, CxcHO, Cd44, Osmr, Cp, Serpina3n, Aspg, Vim and Gfap.

As used herein, “neuronal cell” generally refers to any neuron. In some instances, the methods described herein may inhibit cell death of central nervous system (CNS) neurons, where such CNS neurons will vary and may include, but are not limited to, e.g., cortical neurons, spinal neurons, retinal ganglion cells, cranial nerves, brainstem neurons, cerebellum neurons, diencephalon neurons, cerebrum neurons, and the like.

As used herein, “oligodendrocyte” generally refers to those cells that are a subset of neuroglia that develop from oligodendrocyte precursor cells (OPCs). Oligodendrocytes provide a primary function in myelinating axons of the central nervous system and may be identified by a variety of markers including, but not limited to, e.g., GD3, NG2 chondroitin sulfate proteoglycan, platelet-derived growth factor-alpha receptor subunit (PDGF-alphaR), and the like. Oligodendrocytes, the death of which may be inhibited according to the methods described herein, include immature and mature oligodendrocytes.

The term “subject” refers to any mammalian subject for whom diagnosis, treatment, or therapy is desired, particularly humans. “Mammal” for purposes of the methods described herein refers to any animal classified as a mammal, including humans, domestic and farm animals, and zoo, sports, or pet animals, such as dogs, horses, cats, cows, sheep, goats, pigs, camels, etc. In some embodiments, the mammal is human. In some embodiments, the methods of the application find use in experimental animals, in veterinary application, and in the development of animal models, including, but not limited to, rodents including mice, rats, hamsters, and primates.

Subjects suitable for treatment in accordance with the methods described herein will vary and may include but are not limited to e.g., subjects suspected of having increased levels of neuronal cell death, subjects suspected of having increased levels of oligodendrocyte death, subjects suspected of having increased levels of neuronal and oligodendrocyte cell death, subjects known to have increased levels of neuronal cell death, subjects known to have increased levels of oligodendrocyte death, subjects known to have increased levels of neuronal and oligodendrocyte cell death, subjects suspected of having or known to have increased levels of reactive astrocytes, and the like.

In some instances, subjects suitable for treatment in accordance with the methods described herein include subjects that do not have increased levels of neuronal and/or oligodendrocyte cell death but will be subjected to or otherwise exposed to conditions predicted to cause neuronal and/or oligodendrocyte death. As such, in some instances, the methods described herein include preventing neuron and/or oligodendrocyte cell death in a subject that does not have increased levels of neuronal and/or oligodendrocyte cell death but is expected to be exposed to conditions that increase neuronal and/or oligodendrocyte cell death.

In some embodiments, the condition mediated by reactive astrocytes is a neurodegenerative disease. As used herein, the term “neurodegenerative disease” refers to a disease or condition in which the function of a subject's nervous system becomes impaired.

Accordingly, subjects selected for the methods described herein include those already afflicted with a neurodegenerative disease, as well as those at risk of having a neurodegenerative disease (i.e., in which prevention is desired). Such subjects include those with increased susceptibility to CNS injury, neurodegeneration, or neuroinflammation; those suspected of having CNS injury, neurodegeneration, or neuroinflammation; those with an increased risk of developing CNS injury, neurodegeneration, or neuroinflammation; those with increased environmental exposure to practices or agents causing CNS injury, neurodegeneration, or neuroinflammation, those suspected of having a genetic or behavioral predisposition to CNS injury, neurodegeneration, or neuroinflammation; those with CNS injury, neurodegeneration, or neuroinflammation, those having results from screening indicating an increased risk of CNS injury, neurodegeneration, or neuroinflammation, those having tested positive for a CNS injury, neurodegeneration, or neuroinflammation related condition; those having tested positive for one or more biomarkers of a CNS injury, neurodegeneration, or neuroinflammation related condition, etc.

Exemplary neurodegenerative diseases which subjects may have or be at risk of having for the purposes of the methods described herein include, without limitation, Alzheimer's disease, Parkinson's disease, Huntington's disease, multiple sclerosis, amyotrophic lateral sclerosis, prion disease, motor neurone diseases (MND), spinocerebellar ataxia (SCA), spinal muscular atrophy (SMA), eye-related neurodegenerative disease, e.g., glaucoma, diabetic retinopathy, age-related macular degeneration (AMD), and the like.

In some embodiments, the condition mediated by reactive astrocytes is glaucoma. In some instances, a subject in need of preventing neuronal and/or oligodendrocyte death may be a subject having or at risk of having glaucoma. Such a subject may display one or more symptoms of glaucoma or risk factors for glaucoma including but not limited to e.g., ocular hypertension, above normal ocular pressure (eye pressure of greater than 22 mm Hg), change in vision (including loss of vision), hazy vision, blurred vision, appearance of rainbow-colored circles around bright lights, severe eye pain, head pain, nausea/vomiting accompanying severe eye pain, age over 60 years, family history of glaucoma, steroid use, eye injury, high myopia (nearsightedness), hypertension, central corneal thickness less than 0.5 mm, and combinations thereof.

In another embodiment, the condition mediated by reactive astrocytes is brain cancer. When practicing the methods described herein, the subject may have a brain cancer that includes, without limitation, anaplastic astrocytoma, anaplastic mixed glioma, anaplastic oligodendroglioma, anaplastic oligodendroglioma, germinoma, glioblastoma multiforme, gliosarcoma, low-grade astrocytoma, low-grade mixed oligodendrocyte, low-grade oligodendroglioma, central nervous system lymphoma, medulloblastoma, meningioma, ciliary cell astrocytoma cytoma, acoustic neuroma, chordoma, craniopharynoma, brainstem glioma, ependymoma, optic glioma, epididymal, metastatic brain tumor, pituitary tumor, primitive neuroectodermal, and schwannoma.

In another embodiment, the condition mediated by reactive astrocytes is traumatic brain injury (TBI), e.g., severe TBI, moderate brain injury, mild TBI (MTBI, i.e. concussion), spinal cord injury (SCI), traumatic injury to the eye (including traumatic injury to the nerves of the eye, such as the optic nerve), ischemia, CNS stroke, neuroinflammatory disease, and the like, or acute axonopathy.

In some instances, a subject amendable to treatment as described herein, i.e., a subject suffering from or at risk of suffering from reactive astrocyte mediated neuronal and/or oligodendrocyte death may be a subject having suffered traumatic CNS injury (i.e., CNS neurotrauma). Areas of the CNS that may be injured in a CNS injury include but are not limited to e.g., brain, the spine, etc., as well as neural projections to/from the CNS such as e.g., optic nerves and the like. Non-limiting examples of CNS injuries include traumatic brain injury (TBI), traumatic spinal cord injury (SCI), CNS crush injuries, CNS injuries resulting from a neoplasia (e.g., a brain cancer, e.g., brain tumor), and the like. As used herein, the term CNS injury encompasses injury that occurs as a result of a CNS stroke (e.g., infarct).

In some instances, a subject suffering from reactive astrocyte mediated neuronal and/or oligodendrocyte death and suitable for treatment according to the methods described herein is a subject having suffered a CNS stroke or a subject at increased risk of developing a CNS stroke. The term “stroke” broadly refers to the development of neurological deficits associated with impaired blood flow to the brain regardless of cause. Potential causes include, but are not limited to, thrombosis, hemorrhage and embolism. Current methods for diagnosing stroke include symptom evaluation, medical history, chest X-ray, ECG (electrical heart activity), EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from a tumor, or other blood loss.

In some instances, a subject suitable for treatment in accordance with the methods described herein is a subject having a neuroinflammatory disease or a subject at increased risk of developing a neuroinflammatory disease. Non-limiting examples of neuroinflammatory diseases include acute disseminated encephalomyelitis (ADEM), optic neuritis (ON), transverse myelitis, neuromyelitis optica (NMO) and the like. In some instances, primary conditions with secondary neuroinflammation (e.g., traumatic brain injury with secondary neuroinflammation) may be considered a neuroinflammatory disease as it relates to the subject disclosure.

In another embodiment, the condition mediated by reactive astrocytes is diabetes.

In another embodiment, the condition mediated by reactive astrocytes is leukodystrophy including adrenoleukodystrophy. In some embodiments, the leukodystrophy is a pediatric leukodystrophy. Pediatric leukodystrophy conditions include lysosomal storage diseases (e.g., Tay-Sachs Disease), Cavavan's Disease, Pelizaens-Merzbacher Disease, and Crabbe's Globoid body leukodystrophy.

As used herein, the term “inhibit” or “inhibiting” refers to the function of a particular agent to effectively impede, retard, arrest, suppress, prevent, decrease, or limit the function or action of reactive astrocytes to mediate neuronal and/or oligodendrocyte cell death. In such instances an agent that inhibits is referred to as an “inhibitor”, which term is used interchangeably with “inhibitory agent” and “antagonist”. As used herein, the term “inhibitor” refers to any substance or agent that interferes with or slows or stops a chemical reaction, a signaling reaction, or other biological or physiological activity. An inhibitor may be a direct inhibitor that directly binds the substance or a portion of the substance that it inhibits or it may be an indirect inhibitor that inhibits through an intermediate function, e.g., through binding of the inhibitor to an intermediate substance or agent that subsequently inhibits a target.

Inhibitors contemplated for use in the methods of the present application, i.e., inhibitors of ELOVL1 and lipoapoptosis, include, without limitation, small molecules, oligonucleotides, antibodies or antibody fragments, aptamers, peptides, and inhibitory nucleic acid molecules such as siRNA, antisense oligonucleotide, and microRNA

In some embodiments, the method of inhibiting reactive astrocyte mediated neuronal and/or oligodendrocyte cell death is achieved by administering an inhibitor Elongation of Very Long Chain Fatty Acids Protein 1 (ELOVL1). An ELVOVL1 inhibitor includes any molecule or agent that decreases the activity of ELOVL1 by at least 10%, at least 20%, at least 30%, at least 40%, at least 50%, at least 60%, at least 70%, at least 80%, at least 90%, at least 95%, at least 99%, or even 100% (i.e., no activity) compared to the activity of ELOVL1 in the absence of an inhibitor.

ELOVL1 is a component of the long-chain fatty acids elongation cycle. It is a transmembrane enzyme in the endoplasmic reticulum that adds two carbons per cycle to long- and very long-chain fatty acids with a preference for condensing saturated C18 to C26 acyl-CoA substrates, with the highest activity towards C22:0 acyl-CoA (Ohno et al., “ELOVL1 Production of C24 Acyl-CoAs is Linked to Sphingolipid Synthesis,” Proc Natl Acad Sci 107:18439-44 (2010), which is hereby incorporated by reference in its entirety).

A variety of ELOVL1 inhibitors that are known in the art as suitable for use in accordance with the methods described herein. in one embodiment, the ELOVL1 inhibitor comprises rapamycin, a derivative, or analog thereof (Guo et al., “Rapamycin Inhibits Expression of Elongation of Very-long-chain Fatty Acids 1 and Synthesis of Docosahexaenoic Acid in Bovine Mammary Epithelial Cells,” Asian-Australas J Anim Sci 29(11):1646-1652 (2016), which is hereby incorporated by reference in its entirety).

In another embodiment, the ELOVL1 inhibitor comprises a fibrate or a derivative or analog thereof as described in Schackmann et al., “Enzymatic Characterization of ELOVL1, a Key Enzyme in Very Long-Chain Fatty Acid Synthesis,” Biochimica et Biophysica Acta Molecular and Cell Biology of Lipids 1851(2):231-237 (2015), which is hereby incorporated by reference in its entirety. The fibrate may include bezafibrate or an ester thereof, or gemfibrozil or an ester thereof (Schackmann et al., “Enzymatic Characterization of ELOVL1, a Key Enzyme in Very Long-Chain Fatty Acid Synthesis,” Biochimica et Biophysica Acta Molecular and Cell Biology of Lipids 1851(2):231-237 (2015), which is hereby incorporated by reference in its entirety).

In other embodiments, the ELOVL1 inhibitor comprises oleic acid, a derivative or analog thereof, erucic acid, a derivative or analog thereof, a mixture of oleic acid and erucic acid, or a 4:1 mixture of oleic acid and erucic acid (Lorenzo's oil) (Sassa et al., “Lorenzo's Oil Inhibits ELOVL1 and Lowers the Level of Sphinogomyelin with a Saturated Very Long-chain Fatty Acid,” J Lipid Res 55(3):524-30 (2014), which is hereby incorporated by reference in its entirety).

In another embodiment, the ELOVL1 inhibitor is a nucleic acid molecule inhibitor, e.g., an antisense oligonucleotide, an siRNA, a microRNA, etc. In some embodiments, the inhibitory nucleic acid molecule comprises miR-196a as described in Shah et al., “MicroRNA Profiling Identifies miR-196a as Differentially Expressed in Childhood Adrenoleukodystrophy and Adult Adrenomyeloneuropathy,” Mol. Neurobiol. 54(2):1392-1402 (2017), which is hereby incorporated by reference in its entirety.

In some embodiments, the method of inhibiting reactive astrocyte mediated neuronal and/or oligodendrocyte cell death is achieved by administering an inhibitor of lipoapoptosis. Lipoapoptosis is apoptosis caused by exposure to an excess of fatty acids. An inhibitor of lipoapoptosis is any molecule or agent that inhibits, directly or indirectly, any step in the process of cell death mediated by saturated lipids. As described herein, in this pathway, saturated lipids activate the PERK (Protein Kinase R-like ER Kinase) endoplasmic reticulum stress response pathway, leading to cell death via PUMA (p53 upregulated modulator of apoptosis, Bbc3) driven by the dephosphorylation of Foxo3a (Cunha et al., “Death Protein 5 and p53-Upregulated Modulator of Apoptosis Mediate the Endoplasmic Reticulum Stress-Mitochondrial Dialog Triggering Lipotoxic Rodent and Human β-cell Apoptosis,” Diabetes 61:2763-2775 (2012), which is hereby incorporated by reference in its entirety) followed by Caspase 3 cleavage and cell death (see Examples described herein). The inhibitor may be a general inhibitor of lipoapoptosis, or the inhibitor may inhibit specific pathways of induction. Inhibitors that target multiple steps in the process of lipoapoptosis are also contemplated for use herein.

In one embodiment, the inhibitor of lipoapotosis is an inhibitor of p53 upregulated modulator of apoptosis (PUMA). PUMA inhibitors contemplated for use in the methods of the present application include inhibitors which block PUMA itself as well as its upstream and downstream targets. PUMA is a transcriptional target of p53 and a mediator of DNA damage-induced apoptosis (Mustata et al., Development of Small-molecule PUMA Inhibitors for Mitigating Radiation-induced Cell Death,” Curr. Top. Med. Chem. 11(3):281-290 (2012), which is hereby incorporated by reference in its entirety). PUMA is transcriptionally activated by a wide range of apoptotic stimuli and transduces these proximal death signals to the mitochondria. In particular, PUMA directly binds to all five known anti-apoptotic Bcl-2 family members with high affinities through its BH3 domain. Binding of PUMA to the Bcl-2 like proteins results in the displacement of the proteins Bax/Bak. This displacement results in the activation of Bax/Bak via formation of multimeric pore like structures on the mitochondrial outer membrane, leading to mitochondrial dysfunction and caspase activation. Thus, for the purposes of the methods described herein, inhibitors which disrupt the interaction of PUMA with Bcl-2 proteins are contemplated. Mustata et al., “Development of Small-molecule PUMA Inhibitors for Mitigating Radiation-induced Cell Death,” Curr. Top. Med. Chem. 11(3):281-290 (2012), which is hereby incorporated by reference in its entirety, describes several small molecule inhibitors of PUMA and Bcl-2 family proteins as shown in Table 1 below, which can be utilized in the methods described herein.

TABLE 1
PUMA Inhibitors*

| Compound identifier | Compound Structure |
|---|---|
| 19618875 | 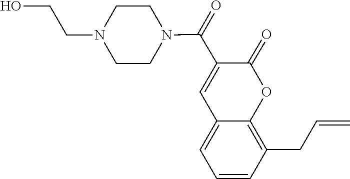 [SMILES: C=CCc1cccc2cc(C(=O)N3CCN(CCO)CC3)c(=O)oc12] |
| 19781680 | 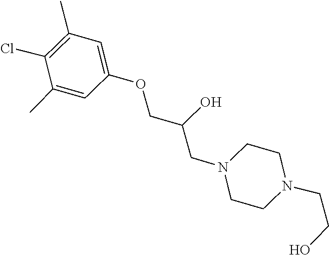 [SMILES: Cc1cc(OCC(O)CN2CCN(CCO)CC2)cc(C)c1Cl] |
| 19882186 | 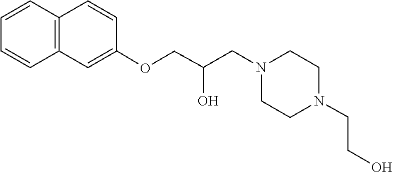 [SMILES: OCCN1CCN(CC(O)COc2ccc3ccccc3c2)CC1] |
| 19899352 | 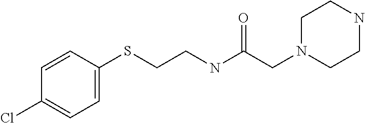 |
| 19957969 | 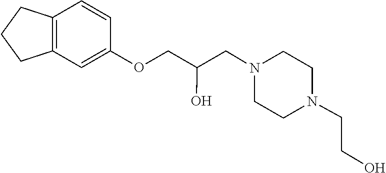 [SMILES: OCCN1CCN(CC(O)COc2ccc3c(c2)CCC3)CC1] |
| 20707440 | 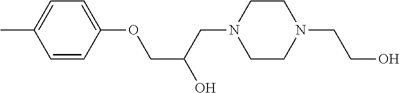 [SMILES: Cc1ccc(OCC(O)CN2CCN(CCO)CC2)cc1] |
| 20719463 | 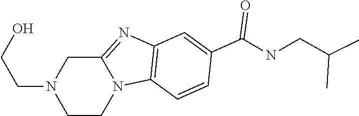 |
| 20732753 | 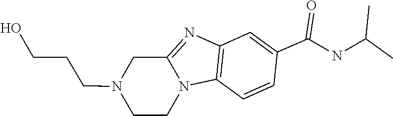 |
| 12909935 | 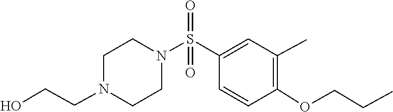 [SMILES: CCCOc1ccc(S(=O)(=O)N2CCN(CCO)CC2)cc1C] |
| 23338424 | 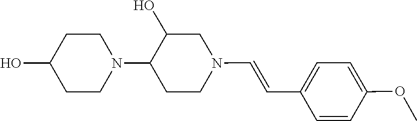 [SMILES: COc1ccc(/C=C/N2CCC(N3CCC(O)CC3)C(O)C2)cc1] |

*PUMA inhibitors are identified in Mustata et al., Curr. Top. Med. Chem. 11(3):281-290 (2012), which is hereby incorporated by reference in its entirety.

Other exemplary PUMA inhibitors are known in the art and include, without limitation, CLZ-8 having the following structure

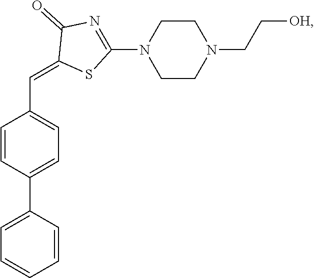 [SMILES: O=C1N=C(N2CCN(CCO)CC2)S/C1=C\c1ccc(-c2ccccc2)cc1]

or an analog or derivative thereof (Feng et al., “CLZ-8, A Potent Small-Molecule Compound, Protect Radiation-Induced Damages Both In vitro and In vivo,” Environ. Tox. Pharm. 61:44-51 (2018), which is hereby incorporated by reference in its entirety).

In some instances, the inhibitor of the present disclosure may be administered directly, e.g., surgically or by injection, to an area behind the blood brain barrier (BBB). In other instances, the inhibitor may be formulated to cross the BBB and thus make direct administration unnecessary. In certain circumstances, neither direct administration within the BBB nor functionalization of the inhibitor to cross the BBB is necessary due to exposure of the underlying target neural tissue or permeabilization of the BBB. Exposure of the underlying target neural tissue and/or permeabilization of the BBB may result as a consequence of the specific condition or incidence from which a subject's condition is a result or may be purposefully caused as a means of administering the inhibitor. In some instances, exposure to trauma, e.g., traumatic brain injury or other CNS trauma (e.g., spinal cord injury, concussion, ischemia, etc.), may permeabilize the BBB allowing delivery across the BBB of an inhibitor that is not functionalized to cross the BBB nor is directly delivered within the BBB. Conditions where the BBB of a subject is permissive to delivery of an inhibitor including inhibitors that have not been functionalized to cross the BBB may be determined by the ordinary skilled medical practitioner upon observation of the subject.

Alternatively, in practicing the methods of the present application, suitable modes of systemic administration include, without limitation orally, topically, transdermally, parenterally, intradermally, intramuscularly, intraperitoneally, intravenously, subcutaneously, or by intranasal instillation, by intracavitary or intravesical instillation, intraocularly, intraarterialy, intralesionally, or by application to mucous membranes. Suitable modes of local administration include, without limitation, catheterization, implantation, direct injection, dermal/transdermal application, or portal vein administration to relevant tissues, or by any other local administration technique, method or procedure generally known in the art. The mode of affecting delivery of the inhibitor will vary depending on the type of inhibitor.

The inhibitor may be orally administered, for example, with an inert diluent, or with an assimilable edible carrier, or it may be enclosed in hard or soft shell capsules, or it may be compressed into tablets, or they may be incorporated directly with the food of the diet. The inhibitor may also be administered in a time release manner incorporated within such devices as time-release capsules or nanotubes. Such devices afford flexibility relative to time and dosage. For oral therapeutic administration, the inhibitor may be incorporated with excipients and used in the form of tablets, capsules, elixirs, suspensions, syrups, and the like. Such compositions and preparations should contain at least 0.1% of the inhibitor, although lower concentrations may be effective and indeed optimal. The percentage of the inhibitor in these compositions may, of course, be varied and may conveniently be between about 2% to about 60% of the weight of the unit.

When the inhibitor is administered parenterally, solutions or suspensions of the inhibitor can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations of the inhibitor suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

In addition to the formulations described previously, the inhibitor may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

According to the methods as described herein, an effective amount of an inhibitor described herein may be administered to a subject, e.g., a subject having a condition as described herein or at risk for having a condition as described herein. In some instances, an effective dose may be the human equivalent dose (HED) of a dose administered to a mouse, e.g., a twice daily dose administered to a mouse. In some instances, the total amount contained in twice daily doses may be administered once daily.

Treatments described herein may be performed chronically (i.e., continuously) or non-chronically (i.e., non-continuously) and may include administration of an inhibitor chronically (i.e., continuously) or non-chronically (i.e., non-continuously). Chronic administration of an inhibitor according to the methods described herein may be employed in various instances, including e.g., where a subject has a chronic condition, including e.g., a chronic neurodegenerative condition (e.g., Alzheimer's disease, Huntington's disease, Parkinson's disease, amyotrophic lateral sclerosis, etc.). Administration of an inhibitor for a chronic condition may include, but is not limited to, administration of the inhibitor for multiple months, a year or more, multiple years, etc. Such chronic administration may be performed at any convenient and appropriate dosing schedule including but not limited to e.g., daily, twice daily, weekly, twice weekly, monthly, twice monthly, etc. Non-chronic administration of an inhibitor may include, but is not limited to, e.g., administration for a month or less, including e.g., a period of weeks, a week, a period of days, a limited number of doses (e.g., less than 10 doses, e.g., 9 doses or less, 8 doses or less, 7 doses or less, etc., including a ingle dose).

An effective amount of a subject compound will depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject being treated.

Therapeutically effective doses of a subject compound or pharmaceutical composition can be determined by one of skill in the art, with a goal of achieving local (e.g., tissue) concentrations that are at least as high as the IC₅₀ of an applicable compound disclosed herein. The specific dose level and frequency of dosage for any particular subject may be varied and will depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

### EXAMPLES

The examples below are intended to exemplify the practice of embodiments of the disclosure but are by no means intended to limit the scope thereof.

### Materials and Methods

### Animals

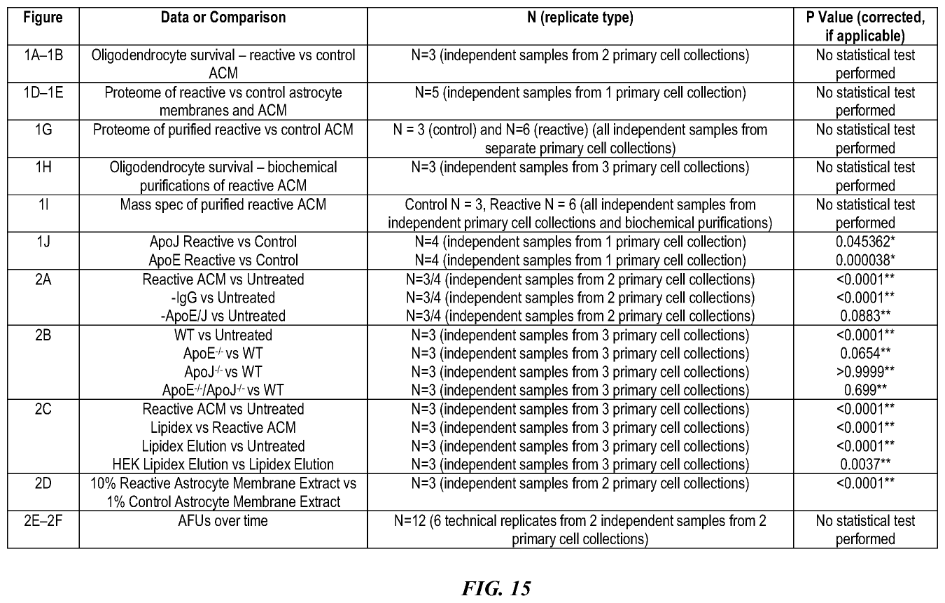

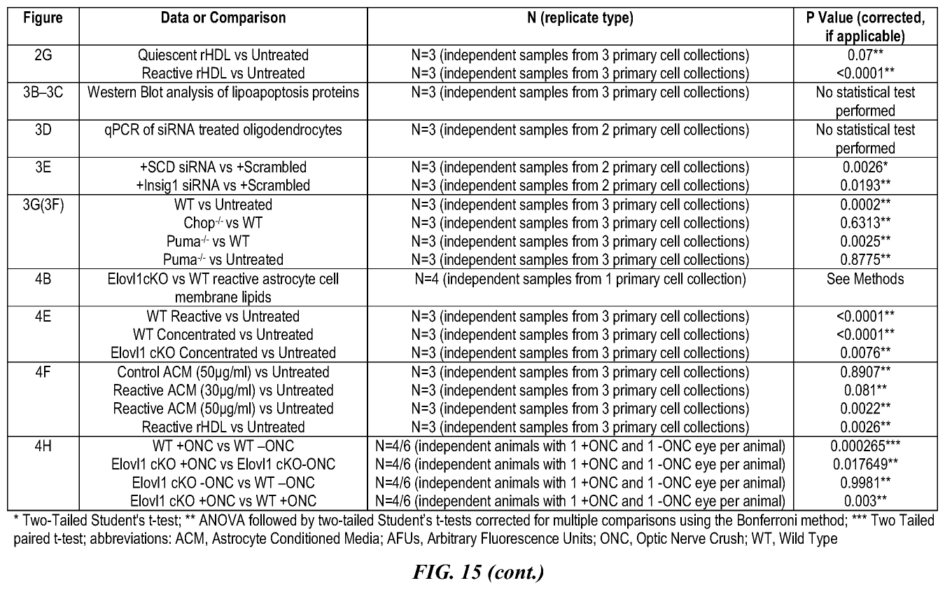

All animal procedures were conducted in accordance with guidelines from the National Institute of Health and Stanford University's Administrative Panel on Laboratory Animal Care or the Institutional Animal Care and Use Committee of NYU Grossman School of Medicine. All animals were housed with food and water available ad libitum in a 12 hour light/dark environment at 20-22° C. and 30-70% humidity. Puma^{-/-} (C57BL/6-Bbc3tm1 Asta; Jax 011067), Chop^{-/-} (B6.129S(Cg)-Ddit3tm2.1Dron/J; Jax 005530), ApoE^{-/-} (B6.129P2-Apoetm1Unc/J; Jax 002052), ApoJ^{-/-} (B6.Cg-Clutm1Jakh/J; Jax 005642), and NuTrap (B6;129S6-Gt(ROSA)26Sor^{tm2(CAG-NuTRAP)Evdr}/J, Jax 029899) mice were obtained from Jax. Sprague Dawley rats were obtained through Charles River (Strain 400). Elovl1^{flox/flox} were generated by Merck & Co., Inc. (Kenilworth, NJ, USA), obtained through Taconic Biosciences (Taconic 10906), and bred into the B6.Cg-Tg(Gfap-cre)77.6Mvs/2J line (Jax 024098). Mixed gender animals were used for all experiments. Postnatal day 5 (P5) mice and P6 rats were used for primary cell isolation. Optic nerve crush experiments performed on P30-P50 mice. All animal studies were performed on animals from different 2 different litters over many months. Number of separate replications for each experiment available in FIG. 15. For all experiments, all attempts at replication were successful. Mice were randomly selected within genotype for assignment to different experiments. All mice were given a number after birth and subsequent experiments performed blind to age and genotype. Sample sizes were determined by reference to previous literature, with samples sizes for optic nerve crush and in vitro experiments determined according to Liddelow et al., “Neurotoxic Reactive Astrocytes are Induced by Activated Microglia,” Nature 541(7638):481-487 (2017), which is hereby incorporated by reference in its entirety.

### Immunopanning and Cell Culture Methods

Astrocytes: Astrocytes were purified by immunopanning from P5 mice or P6 Sprague Dawley rat forebrains and cultured as previously described (Foo et al., “Development of a Method for the Purification and Culture of Rodent Astrocytes,” Neuron 71(5):799-811 (2011), which is hereby incorporated by reference in its entirety). Cortices were blunt dissected and enzymatically digested using papain at 37° C. and 10% CO₂. Tissue was then mechanically triturated with a 5 mL serological pipette at room temperature to generate a single-cell suspension. The suspension was filtered in a 70 μm nitex filter and negatively panned for microglia (CD45; BD Pharmingen 554875 for mouse, BD Pharmingen 553076 for rat), endothelial cells (BSL I, Vector Labs L-1100), and oligodendrocyte lineage cells (O4 hybridoma, in house) followed by positive panning for astrocytes (for mouse: HepaCAM, R&D Systems MAB4108; for rat: ITGB5, Thermo, 14-0497-80). Astrocytes were removed from the final positive selection plate by brief digestion with 0.025% trypsin and plated on poly-d-lysine coated tissue culture plates. Astrocytes were cultured in defined, serum-free medium containing 50% neurobasal, 50% DMEM, 100 U/mL penicillin, 100 μg/mL streptomycin, 1 mM sodium pyruvate, 292 μg/mL L-glutamine, 1×SATO, 5 μg/mL of N-acetyl cysteine, and 5ng/mL HBEGF (Peptrotech, 100-47).

For collection of astrocyte conditioned media, plates of astrocyte cultures from identical preps were randomly chosen as control or reactive. Reactive astrocyte cultures were treated for 24 hours with IL1α (3 ng/ml, Sigma, 13901), TNF (30 ng/ml, Cell Signaling Technology, 8902SF), and C1q (400 ng/ml, MyBioSource, MBS143105). Control and reactive astrocyte conditioned media (ACM) was collected and spun at ˜2000 g for 5 minutes to remove any dead cells or cell debris. ACM was then concentrated in a Vivaspin 30 kDa centrifugation tubes (Cytiva 28932361) to ˜10× concentration for subsequent experiments. The protein concentration of ACM was determined by Bradford Assay (Sigma-B6916) and used to ensure identical concentrations of reactive versus control ACM were used for further experiments. ACM was presented at a dose of 50 μg/ml in all experiments not otherwise denoted with an alternative concentration.

Oligodendrocytes: Oligodendrocyte lineage cells were purified by immunopanning from P6 Sprague-Dawley forebrains and cultured as previously described (Dugas and Emery, “Purification of Oligodendrocyte Precursor Cells from Rat Cortices by Immunopanning,” Cold Spring Harbor Protocols 2013(8):745-758 (2013), which is hereby incorporated by reference in its entirety). Cortices were blunt dissected and enzymatically digested using papain at 37° C. and 10% CO₂. Tissue was then mechanically triturated with a 5 mL serological pipette at room temperature to generate a single-cell suspension. The suspension was filtered in a 70 μm nitex filter and negatively panned for astrocytes (Ran2 hybridoma; in house (Dugas and Emery, “Purification of Oligodendrocyte Precursor Cells from Rat Cortices by Immunopanning,” Cold Spring Harbor Protocols 2013(8):745-758 (2013), which is hereby incorporated by reference in its entirety) and mature oligodendrocytes (GalC hybridoma; in house (Dugas and Emery, “Purification of Oligodendrocyte Precursor Cells from Rat Cortices by Immunopanning,” Cold Spring Harbor Protocols 2013(8):745-758 (2013), which is hereby incorporated by reference in its entirety)) followed by positive panning for oligodendrocyte progenitor cells (OPCs; O4 hybridoma; in house (Dugas and Emery, “Purification of Oligodendrocyte Precursor Cells from Rat Cortices by Immunopanning,” Cold Spring Harbor Protocols 2013(8):745-758 (2013), which is hereby incorporated by reference in its entirety)). OPCs were removed from the final positive selection plate by brief digestion with 0.025% trypsin and plated on poly-d-lysine coated tissue culture plates. OPCs were cultured in defined, serum-free proliferation medium for 48 hours containing DMEM with 100 U/mL penicillin, 100 μg/mL streptomycin, 1 mM sodium pyruvate, 292 μg/mL L-glutamine, 1× SATO, 5 μg/mL of N-acetyl cysteine, 5 μg/ml insulin, 1× Trace elements B (Cellgro 99-175-CI), 10 ng/ml d-Biotin (Sigma B4639), 10 ng/ml PDGF (Pepro-tech 100-13A), 4.2 μg/ml Forskolin (Sigma F6886), 10 ng/ml CNTF (Peprotech 450-02), and 1 ng/ml NT-3 (peprotech 450-03). OPCs were then plated in defined, serum-free differentiation medium containing DMEM with 100 U/mL penicillin, 100 μg/mL streptomycin, 1 mM sodium pyruvate, 292 μg/mL L-glutamine, 1× SATO, 5 μg/mL of N-acetyl cysteine, 5 μg/ml insulin, 1× Trace elements B (Cellgro 99-175-CI), 10 ng/ml d-Biotin (Sigma B4639), 4.2 μg/ml Forskolin (Sigma F6886), 10 ng/ml CNTF (Peprotech 450-02), and 40 ng/ml T3 (Sigma T6397). Cells were 50% media changed every 48 hours until experiments were complete. Mature oligodendrocyte experiments performed beginning 3 days after transfer to differentiation media.

Retinal ganglion cells: Retinal ganglion cells were isolated from P5-7 Sprague Dawley rat retinas as previously described (Ullian et al., “Control of Synapse Number by Glia,” Science 291(5504):657-661 (2001), which is hereby incorporated by reference in its entirety). RGCs were plated on glass coverslips (12 mm diameter, Carolina Biological Supply 633029) coated with poly-D-lysine (Sigma P6407) and laminin (R&D 340001001) at a density of 30,000 cells/well in media containing 50% DMEM (Thermo Fisher Scientific 11960044), 50% Neurobasal (Thermo Fisher Scientific 21103049), Penicillin-Streptomycin (LifeTech 15140-122), glutamax (Thermo Fisher Scientific 35050-061), sodium pyruvate (Thermo Fisher Scientific 11360-070), N-acetyl-L-cysteine (Sigma A8199), insulin (Sigma 11882), triiodo-thyronine (Sigma T6397), SATO (containing: transferrin (Sigma T-1147), BSA (Sigma A-4161), progesterone (Sigma P6149), putrescine (Sigma P5780), sodium selenite (Sigma S9133)), B27 (see (Winzeler and Wang, 2013) for recipe), BDNF (Peprotech 450-02), CNTF (Peprotech 450-13), and forskolin (Sigma F6886). RGC cultures were maintained in a humidified incubator at 37° C. and 10% CO₂ for 7 days before treatment.

HEK293T Cells: HEK293 cells were cultured in DMEM (GIBCO, 11960044) with 10% fetal bovine serum (FBS; GIBCO, 16000044), 2 mM L-glutamine (GIBCO, 25030081), 1 mM sodium pyruvate (GIBCO, 11360070), and 1,000 U/ml Penicillin-Streptomycin (GIBCO, 15140148). Cells were cultured in a 37° C. humidified incubator containing 5% CO₂. HEK293T cells were not authenticated after purchase or tested for mycoplasma contamination. A fully confluent 10 cm plate was used for collecting cell membranes and conditioned media for experiments.

### Live/Dead Analysis

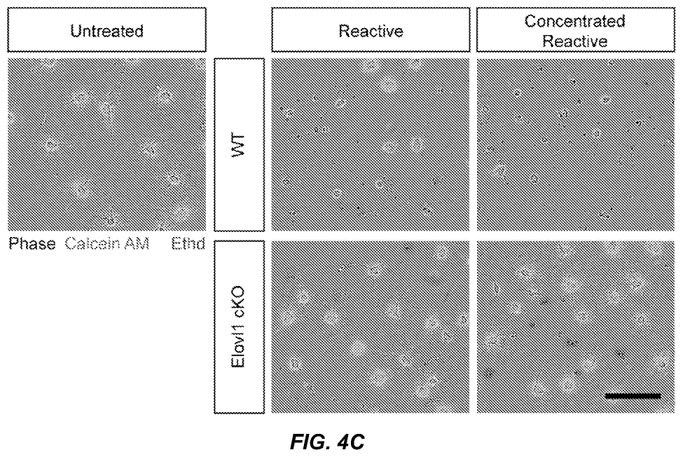

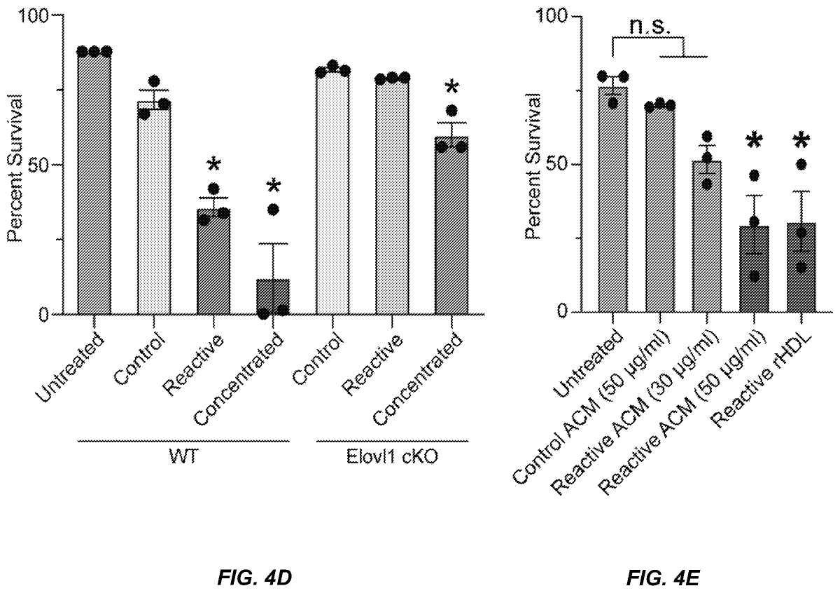

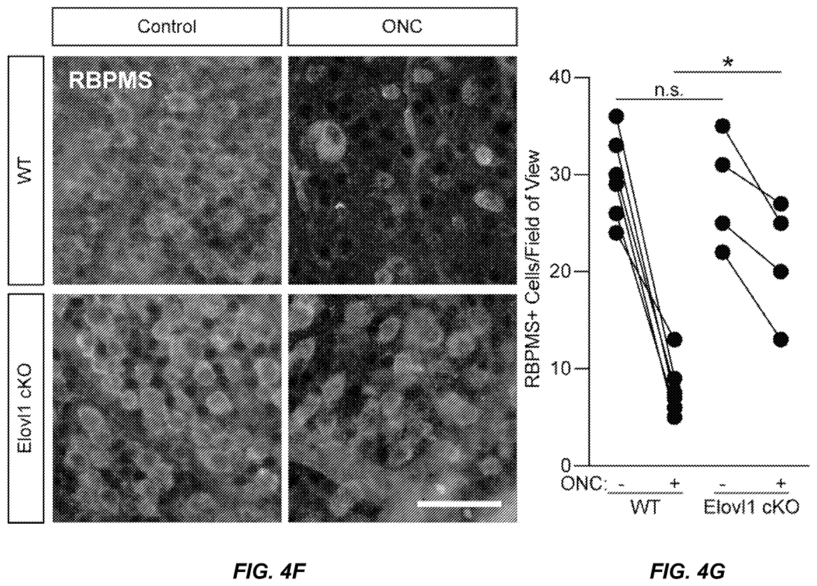

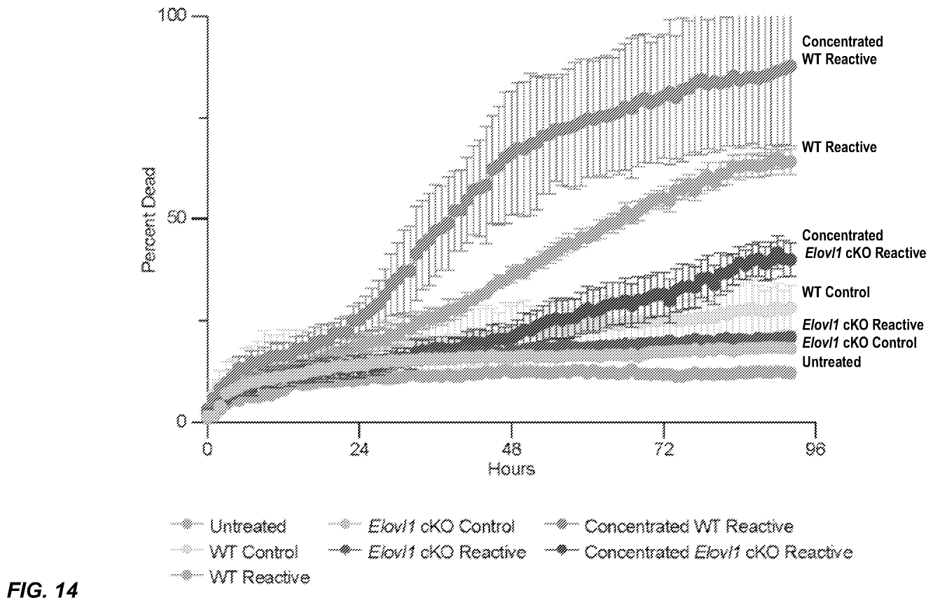

RGC were cultured for 7 days prior to treatment and mature oligodendrocytes were treated 3 days after exposure to differentiation medium. All experiments began with identically plated cells that were randomly chosen for treatment, ensuring identical starting cell numbers for control and experimental conditions. Live/dead analysis was completed on cells 24 hours after treatment, except for experiments in FIGS. 4D-4E in which cells were treated longer (see FIG. 14) due to lower concentrations of ACM from WT versus cKO mouse astrocytes. In all instances, 3 separate replicates were performed on 3 separate primary cell culturing events to ensure reproducibility. Cells were treated with Calcein AM dye and ethidium homodimer-1 (Invitrogen) at a final concentration of 1.33 μM Calcein AM and 2.5 μM ethidium homodimer-1. Cells were incubated at 37° C. for 10 minutes before imaging on an Axio Observer. Z1 (Zeiss) or on an Incucyte ZOOM (Essen Bioscience). Identical acquisition and illumination conditions were used within each experiment and cells were counted in ImageJ using custom macros, which involved background subtraction and thresholding following by particle counting. Percent survival was defined as the number of Calcein⁺ live cells over the number of Calcein⁺ and Ethidium⁺ total cells. Percent survival was not normalized in the majority of experiments so as not to mask any changes in overall culture viability occurring throughout the experiment. All in vitro experiments were performed at least 3 times from separate individual primary cell preparations.

### Mass Spectrometry Techniques

Global Unbiased Protein Mass Spectometry: The cell lysates were thawed at room temperature and homogenized using the Precellys lysing kit (Bertin Instruments). The bicinchoninic acid assay (BCA) was performed to determine the total protein in each sample, and 20 μg of protein (volume equivalent) from each sample was precipitated overnight at −20° C. with 4× volume of ice-cold acetone. The precipitates were pelleted at 13.5 k RPM for 10 minutes at 4° C., the supernatant discarded, and pellets dried in a vacuum centrifuge for 15 minutes. The dried pellets were then resuspended in a mixture of 15 μL water with 5 μL of 4× Laemmli buffer and subjected to heating at 70° C. for 10 minutes. The samples were separated in the 1 D gel at 200 V for 20 minutes, and the bands on the gel were stained with the Coomassie blue solution for 1 hour. The gels were then rinsed several times with water, and each lane was excised into 6 gel slices for in-gel (Hedrick et al., “Digestion, Purification, and Enrichment of Protein Samples for Mass Spectrometry,” Curr. Protoc. Chem. Biol. 7(3):201-222 (2015), which is hereby incorporated by reference in its entirety). The sliced gel samples were washed 3× times with 25 mM ammonium bicarbonate (ABC) and 50% acetonitrile (ACN), and 1× times with 100% ACN to completely de-stain the gels and dried in a vacuum centrifuge for 15 minutes. Reduction and alkylation of cysteines were carried out using 10 mM dithiothreitol (DTT) in 25 mM ABC at 55° C. for 1 hour and 55 mM iodoacetamide (IAA) in 25 mM ABC at room temperature in the dark for 45 minutes respectively. Dried gel pieces were transferred to Barocycler tubes and digested for 2 hours in a Barocycler at 50° C. and 20,000 psi (50 seconds at 20,000 psi, 10 seconds at atmospheric pressure for a total of 120 cycles or 2 hours) using trypsin/Lys-C mix (Promega #V5071) at an enzyme-to-substrate ratio of 1:25. After digestion, supernatants containing the peptides were removed, and the peptides were extracted using 60% ACN/5% trifluoroacetic acid (TFA). The peptide samples were vacuum dried and re-suspended in 15 μL of sample loading buffer (0.1% (v/v) formic acid in 3% ACN), and 5 μL was used for LC-MS/MS analysis.

The frozen ACM samples were thawed at room temperature, and the BCA was performed to determine the total protein in each sample. 50 μg of protein (equivalent volume) from each ACM sample was precipitated overnight at −20° C. with 4× volume of ice-cold acetone. The next day, the precipitated samples were pelleted at 13.5 k RPM at 4° C. for 10 minutes. The supernatants were discarded, and the precipitated pellets were dissolved in 10 μL of 8M urea containing 10 mM DTT and incubated at 37° C. for 1 hour for reduction. Next, alkylation was performed using 10 μL alkylating reagent (195 μL ACN+1 μL triethylphosphine+4 μL of IAA) by incubating the samples for 1 hour at 37 ° C. The reduced and alkylated samples were then dried in a vacuum centrifuge. For in-solution digestion (Hedrick et al., “Digestion, Purification, and Enrichment of Protein Samples for Mass Spectrometry,” Curr. Protoc. Chem. Biol. 7(3):201-222 (2015), which is hereby incorporated by reference in its entirety), trypsin/Lys-C mix (Promega) was prepared by dissolving the stock reagent in 400 μL of 25 mM ABC. 80 μL of the trypsin/Lys-C mix was added to each sample for digestion in a Barocycler (50° C.; 60 cycles: 50 seconds at 20 kPSI and 10 seconds at 1 ATM). Finally, the peptides were desalted using MicroSpin columns (C18 silica; The Nest Group). The dried, purified peptides were re-suspended in 3% ACN in 0.1% formic acid to a final concentration of 1 μg/μL, and 1 μL was loaded to the HPLC system.

The peptides were analyzed in a Dionex UltiMate 3000 RSLC nano System (Thermo Fisher Scientific, Odense, Denmark) coupled on-line to Orbitrap Fusion Lumos Mass Spectrometer (Thermo Fisher Scientific, Waltham, MA, USA) as described previously (Barabas et al., “Proteome Characterization of used Nesting Material and Potential Protein Sources from Group Housed Male Mice, Mus musculus,” Sci. Rep. 9(1):17524 (2019), which is hereby incorporated by reference in its entirety). Briefly, reverse-phase peptide separation was accomplished using a trap column (300 um ID×5 mm) packed with 5 um 100 Å PepMap C18 medium coupled to a 50-cm long×75 um inner diameter analytical column packed with 2 um 100 Å PepMap C18 silica (Thermo Fisher Scientific). The column temperature was maintained at 50° C. The samples were loaded to the trap column in a loading buffer (3% acetonitrile in 0.1% FA) at a flow rate of 5 μL/min for 5 minutes, and eluted from the analytical column at a flow rate of 200 nL/min using a 160-min LC gradient. The column was washed and equilibrated with three 30-minute LC gradients before injecting the next sample. All data were acquired in the Orbitrap mass analyzer, and the data were collected using an HCD fragmentation scheme. For MS scans, the scan range was from 350 to 1600 m/z at a resolution of 120,000, the automatic gain control (AGC) target was set at 4×10⁵, maximum injection time was 50 ms, dynamic exclusion was 30 seconds, and intensity threshold was 5.0×10⁴. MS data were acquired in the Data Dependent mode with a cycle time of 5 s/scan. The MS/MS data were collected at a resolution of 15,000.

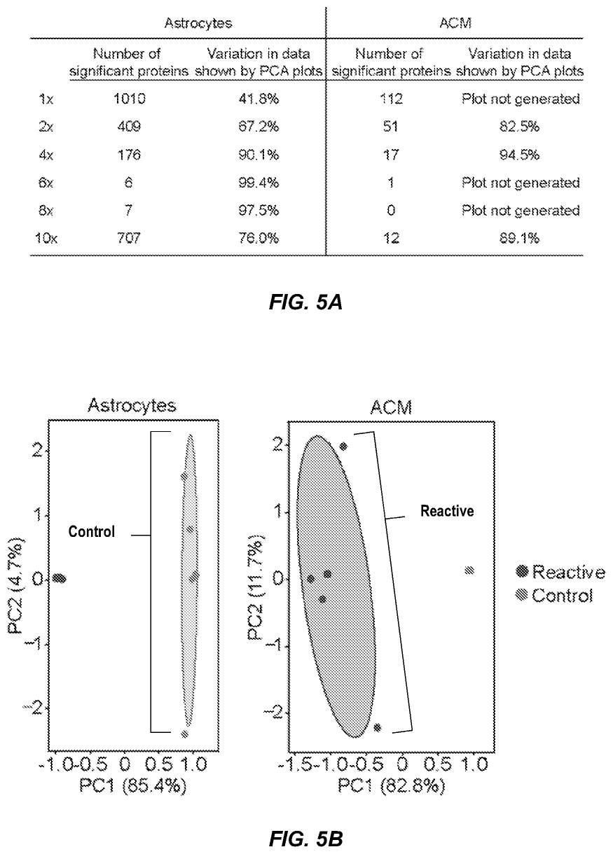

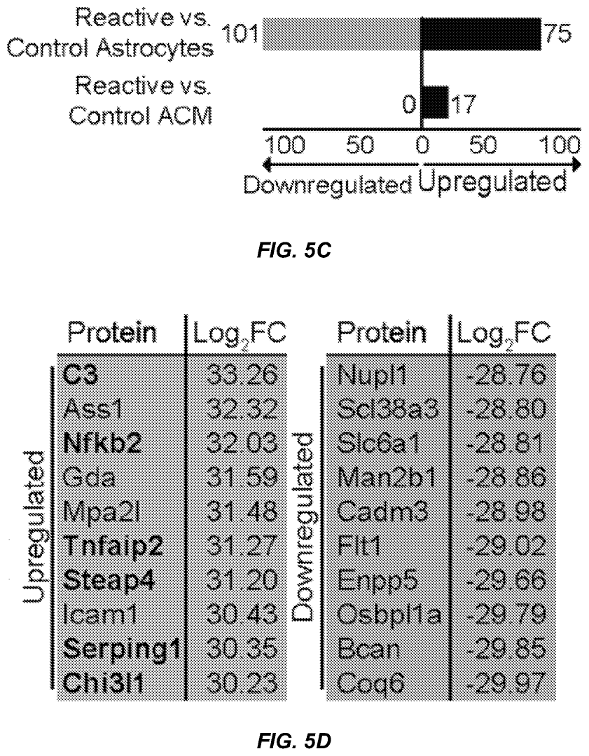

LC-MS/MS data were analyzed using MaxQuant software (version 1.6.3.3) by searching the Rattus norvegicus protein sequence database downloaded from the UniProt in March 2020. The following parameters were edited during search: precursor mass tolerance of 10 ppm; enzyme specificity of trypsin/Lys-C enzyme allowing up to 2 missed cleavages; oxidation of methionine (M) as a variable modification and iodoethanol (C) as a fixed modification. False discovery rate (FDR) of peptide spectral match (PSM) and protein identification was set to 0.01. Proteins with LFQ #0 and MS/MS (spectral counts) ≥2 were only considered as true identification and used for statistical analysis in the Perseus software platform. The edgeR package (McCarthy et al., “Differential Expression Analysis of Multifactor RNA-Seq Experiments with Respect to Biological Variation,” Nucleic Acids Res 40(10):4288-4297 (2012); Robinson et al., “edgeR: A Bioconductor Package for Differential Expression Analysis of Digital Gene Expression Data,” Bioinformatics 26(1):139-140 (2010), which are hereby incorporated by reference in their entirety) in R was used to analyze the data obtained from MaxQuant. Briefly, the label-free quantitation (LFQ) intensity values of the five reactive astrocytes versus the five control astrocytes were used to calculate the log fold change (log₂FC), p-value, and the Benjamini-Hochberg (BH) method to obtain FDR values for each identified protein. The data was analyzed such that each protein had at least 1 non-zero LFQ value, 2 non-zero LFQ values, 4 non-zero LFQ values, 6 non-zero LFQ values, 8 non-zero LFQ values, and 10 non-zero values (1×, 2×, 4×, 6×, 8×, and 10× respectively) between the reactive and control groups. However, 4× data analysis was selected for results and figures based on the total contribution of principal components showcasing greater than 90% variation in reactive and control astrocytes datasets for both cells and ACM (FIGS. 5A-5D). The significant proteins were selected based on FDR<0.1. There is no difference in log₂FC, p-value, FDR between different datasets for the same protein.

### Global Unbiased Lipidomics

Lipid and metabolite extracts from astrocytes and ACM were prepared using a slightly modified Bligh & Dyer extraction procedure (Bligh and Dyer, “Rapid Method of Total Lipid Extraction and Purification,” Can J. Biochem. Phys. 37(8):911-917 (1959), which is hereby incorporated by reference in its entirety). Briefly, the frozen cell pellets were thawed for 10 minutes at room temperature, and 200 μL ultrapure water was added to promote cell lysis, followed by 450 μL methanol and 250 μL HPLC-grade chloroform. The samples were vortexed for 10 seconds, resulting in a one-phase solution, and incubated at 4° C. for 15 minutes. Next, 250 μL ultrapure water and 250 μL chloroform were added, creating a biphasic solution. The samples were centrifuged at 16,000× g for 10 minutes resulting in three phases in the tubes. The bottom organic phase containing the lipids was transferred to new tubes. The middle phase consisting of proteins was discarded, and the upper polar phase containing the metabolites was transferred to separate tubes. The organic and polar phase solvents were evaporated in a vacuum concentrator leaving behind the dried lipid and metabolite extracts. The same protocol was used to extract the lipids and metabolites from the ACM samples. The volumes of the solvents were scaled to 2.5 times per 500 μL of the sample volume.

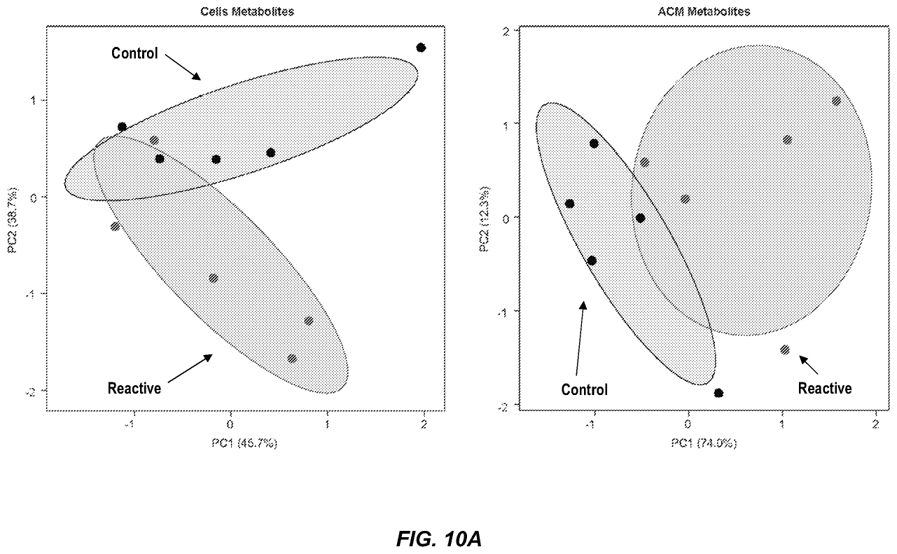

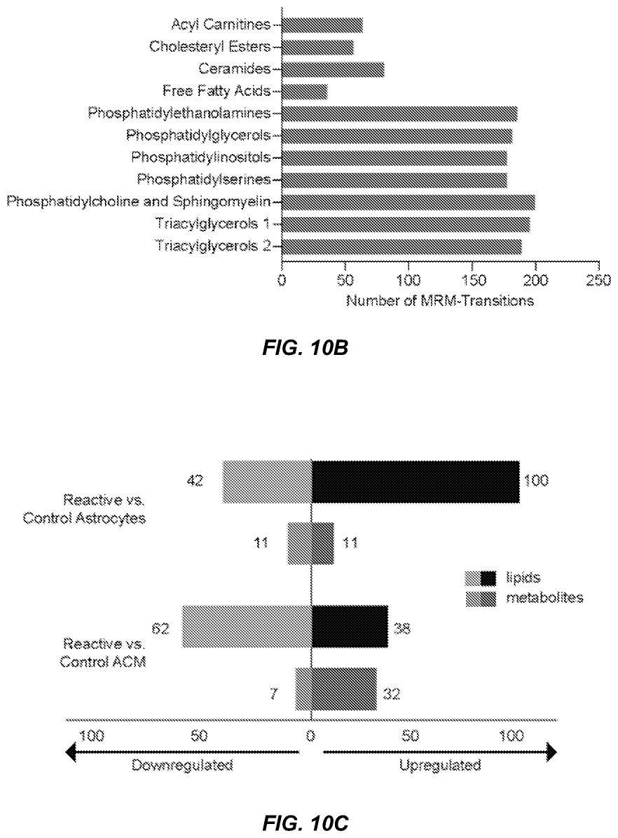

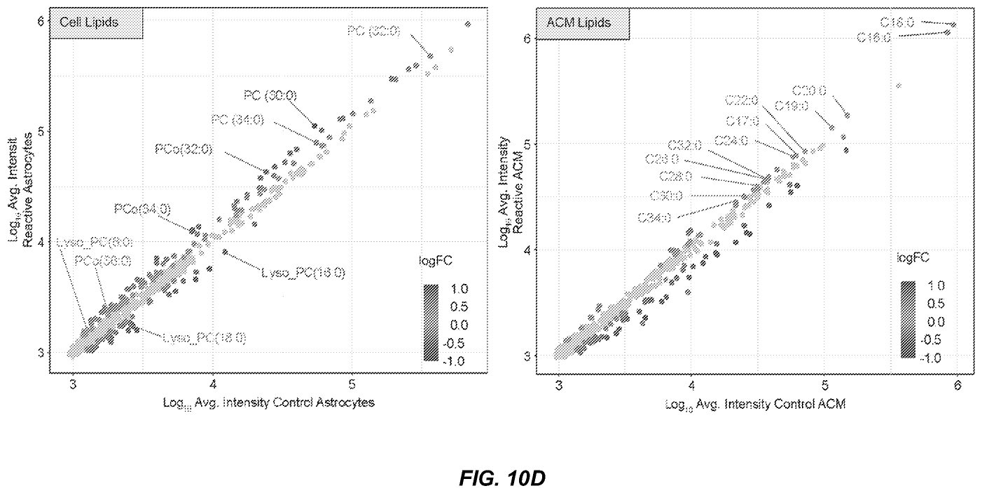

Multiple Reaction Monitoring (MRM)-profiling of the extracted lipids and metabolites was performed as described previously (Xie et al., “Multiple Reaction Monitoring Profiling (MRM profiling): Small Molecule Exploratory Analysis Guided by Chemical Functionality,” Chem. Phys. Lipids 235:105048 (2021), which is hereby incorporated by reference in its entirety). The dried lipid extracts were dissolved in 200 μL methanol:chloroform (3:1 v/v) to make lipid stock solutions and transferred to glass LC vials. The lipids were further diluted 200 times (cells) and 100 times (media) in injection solvent (acetonitrile:methanol:ammonium acetate 300 mM 3:6.65:0.35 (v/v)). The dried metabolites were resuspended in 200 μL (cells) and 1000 μL (media) of MeOH:ACN (1:1 v/v) to make stock solutions. The metabolite stock solutions were diluted 5 times (cells) and 250 times (media) in the injection solvent. The injection solvent alone without any lipids or metabolites was used as the “blank” sample. The injection solvent containing the quantitative mass spectrometry internal standard consisting of a mixture of 13 deuterated lipid internal standards at a concentration of 100 μg/mL each (Avanti Polar Lipids, #330731) was used as the “quality control” sample to monitor their peaks over time to confirm the proper working of the instrument. MS data was acquired by flow-injection (no chromatographic separation) from 8 μL of diluted lipid extract stock solution delivered (per sample per method) using a micro-autosampler (G1377A) to the ESI source of an Agilent 6410 Triple Quadrupole MS. This method enabled the interrogation of the relative amounts of numerous lipid species within ten major lipid classes based on the LipidMaps database. The lipid classes, and the distributions of the total number of MRM transitions screened are presented in FIG. 10B. Triacylglycerides (TAGs) were divided into 2 separate methods (TAG1 and TAG2) based on the fatty acid residues' neutral losses as the product ions. Specifically, TAG1 method screened for 16:0, 16:1, 18:0 and 18:1 fatty acids and TAG 2 method screened for 18:2, 20:0, and 20:4 fatty acids. The raw MS data obtained for lipids and metabolites were analyzed using an in-house script. The lists containing MRM transitions and the respective ion intensity values were exported for statistical analysis.

All statistics for the comparisons of MRM transitions of the lipids and metabolites between reactive astrocytes compared to control astrocytes were calculated using the edgeR package. Here, the ion count for a given molecule (lipid or metabolite) was referred to using the subscript s for the sample (cell replicate for a class of analyte) and b for the specific molecule (lipid or metabolite). An additional ‘intercept’ sample was added to model the experimental blank performed using just the injection media to ensure that all comparisons are significant with respect to this blank control. The edgeR package fits a generalized linear model to the following log-linear relationship for the mean-variance:

logμ_{bs}=X_{b}^{T}β_{g}+log Nₛ

for each molecule b in sample s where the sum of all ion intensity for sample s sums to Nₛ. This allowed for the calculation of the coefficient of variation (CV) for the ion count for a molecule in a sample (γ_{bs}) using the following relationship

CV²(γ_{bs})=1/μ_{bs}+Φ_{b}

where Φ_{b} is the dispersion of the molecule. This dispersion term was estimated using the common dispersion method (McCarthy et al., “Differential Expression Analysis of Multifactor RNA-Seq Experiments with Respect to Biological Variation,” Nucleic Acids Res 40(10):4288-4297 (2012), which is hereby incorporated by reference in its entirety). These values were used to calculate the associated log₂FC between the reactive and control astrocytes and the p-values were obtained using the likelihood ratio test. These p-values were then adjusted for multiple testing using the BH method to FDR. The lipid or metabolite was considered significant when FDR<0.1.

### Targeted Proteomics on Toxic Factor Enrichments

Proteins in solution were precipitated overnight in four volumes of −80° C. acetone, dried, and then resuspended in 50 mM ammonium bicarbonate with 0.01% Protease Max (Promega). Proteins were reduced in 10 mM DTT at 55° C. for 30 minutes, and then alkylated with 30 mM acrylamide for 30 minutes at room temperature. Alkylated proteins were digested overnight using Trypsin/LysC protease (Promega) at 37° C., acidified and dried under SpeedVac prior to LC/MS analysis.

Mass spectrometry experiments were performed using either an Orbitrap Elite or an Orbitrap Fusion Tribrid mass spectrometer (Thermo Scientific, San Jose, CA) with an Acquity M-Class UPLC system (Waters Corporation, Milford, MA) for reverse phase separations. Separations were performed on in-house pulled-and-packed fused silica chromatography columns. The fused silica has an I.D. of 100 microns, and was packed with a C18 reprosil Pur 1.8 micron stationary phase (Dr. Maisch, Germany) to a length of 15-20 cm. The UPLC system was set to a flow rate of 300 nL/min, where mobile phase A was 0.2% formic acid in water and mobile phase B was 0.2% formic acid in acetonitrile. Peptides were directly injected onto the chromatography column, with a gradient of 2-45% mobile phase B, followed by a high-B wash over a total 80 minutes. CID fragmentation was used in a data-dependent fashion for MS/MS spectral generation.

Mass spectra were analyzed using Byonic v 2.6.49 (Protein Metrics) against a UniProt database for Rattus norvegicus containing common contaminants. Precursor mass tolerances were set to 10 ppm with fragment tolerances set to 0.3 Da for CID fragmentation. Peptides were assumed to be semi-tryptic and allowed to have up to two missed cleavages. Various post translational modifications, such as oxidations, methyl, and dimethyl modifications were permitted. Data were validated using the standard reverse-decoy technique at a 1% false discovery rate as described previously (Elias and Gygi, “Target-Decoy search Strategy for Increased Confidence in Large-Scale Protein Identifications by Mass Spectrometry,” Nat. Methods 4(3):207-214 (2007), which is hereby incorporated by reference in its entirety). In-house tools were used for further data analysis and visualization.

### Toxic Factor Enrichments

ACM was collected from 10×10 cm plates of immunopanned astrocytes made reactive by treatment with IL-1α, TNFα, and C1q (see immunopanning and cell culture), centrifuged at 500× g for 5 minutes to eliminate floating debris, and treated with Roche complete protease inhibitor (Millipore 5892791001). ACM was first concentrated ˜10× using Vivaspin 30 kDa centrifugation tubes (Cytiva 28932361) and then loaded on to an anion exchange (HiTrap Q High Performance; Cytiva GE17-1153-01), cation exchange (HiTrap Sp High Performance; Cytiva GE17-1151-01), or hydrophobic interaction (HiTrap Phenyl Fast Flow (LS); Cytiva GE17-5194-01) columns. Anion and cation exchange columns were eluted in order with HEPES buffered pH 7.5 solutions of 0M NaCl, 0.25M NaCl, 0.5M NaCl, 0.75M NaCl, and 1M NaCl according to manufacturer's instructions and each fraction concentrated to the same final volume using vivaspin 30 kDa centrifugation tubes (Cytiva 28932361). Hydrophobic interaction columns were eluted in order with HEPES buffered pH 7.5 solutions of 1M NaCl, 0.75M NaCl, 0.5M NaCl, 0.25M NaCl, and 0M NaCl according to manufacturer's instructions and each fraction concentrated to the same final volume using Vivaspin 30 kd centrifugation tubes (Cytiva 28932361). Ammonium sulfate precipitation was performed by adding fully saturated ammonium sulfate to the ACM dropwise while vortexing until the desired percent saturation was achieved. The solution was then centrifuged at 4,000× g for 10 minutes and the supernatant carefully decanted. This process was repeated in serial until the pellet was removed from the ACM at 10%, 20%, 30%, 40%, 50%, 60%, 70% ammonium sulfate saturation. All pellets and supernatant were then desalted in a pH 7.5 HEPES buffer using progressive dilution and concentration with Vivaspin 30 kDa centrifugation tubes until ≥100× dilution of ammonium sulfate was achieved and all fractions were concentrated to the same final volume.

To test for toxicity, identical volumes of each of the above fractions were added to oligodendrocytes at an identical final concentration, determined by the toxic activity of the starting toxic ACM, and live dead analysis performed at 24 hours. For FIG. 4E, ACM was concentrated 10 fold using a Vivaspin 30 kDa centrifugation tube.

For final mass spec analysis, toxic or control ACM was first concentrated 10× using Vivaspin 30 kDa centrifugation tubes. The concentrated ACM was then loaded on the above listed cation exchange column and washed with 0M NaCl and the flowthrough collected. This flowthrough was then loaded on to the above listed anion exchange column and washed with 0 M NaCl and the flowthrough collected. The above flowthrough was then loaded onto the above listed hydrophobic interaction chromatography column, which was washed with 0.75 M NaCl (discarded) and eluted with 1 M NaCl (collected). This elution was desalted by progressive dilution and concentration with a pH 7.5 HEPES buffer and Vivaspin 30 kDa centrifugation tubes. The solution was then raised to 60% ammonium sulfate precipitation using the above listed technique and the pellet desalted and concentrated to 100 μl final volume. The resultant toxic factor enrichments were subjected to protein mass spectrometry analysis.

High Performance Liquid Chromatographic Separation of Lipoproteins from Conditioned Media

200 μl of conditioned media from either control or reactive astrocytes was injected onto two Superose 6 Increase 10/300 GL columns (Cytiva, MA, USA) in tandem. Lipoproteins were separated by size exclusion chromatography. Fractions were collected, with fractions 60-70 representing the size of high-density lipoproteins (HDL). The absorbance at 280 nm was measured to determine protein concentration of each fraction. Eluted fractions were stored at −80 until further analysis.

Quantification of ApoJ/Clusterin and ApoE from HPLC fractions

Apolipoprotein J (ApoJ)/Clusterin and ApolipoproteinE (ApoE) were quantified in fractionated conditioned media from either reactive or control samples by enzyme-linked immunosorbent assay (ELISA). ApoJ/Clusterin was quantified using a rat ApoJ/Clusterin ELISA (Thermo Fisher Scientific, Waltham, MA) following the manufacturer's instructions. ApoJ/Clusterin was measured in whole conditioned media, and all undiluted fractions to detect the size range in which ApoJ/Clusterin was present. ApoJ/Clusterin was then quantified in fractions within the HDL size range for all samples. ApoE was quantified using a rat ApoE ELISA (Elabscience Biotechnology Co., Wuhan, China), following the manufacturer's instructions. Fractions from the HDL range were pooled for each sample and concentrated using Pierce protein concentrators (Thermo Fisher Scientific, Waltham, MA). ApoE was measured in concentrated samples within the HDL size range.

### Protein and Lipid Depletion and Presentation

Antibody pulldown were performed using the Dynabeads Antibody Coupling Kit (Thermo, 14311D) according to manufacturer's protocols using antibodies against ApoE (Fisher, 701241) and ApoJ (US Biological Life Sciences, 139770) or Rabbit IgG control (Abcam, ab172730) and (Abcam, ab37373). Pulldowns were performed on ACM for 4 hours at room temperature on a Tube Rotator and Rotisseries (VWR, 10136-084) with vortexing every 30 minutes. Lipid depletion from identically concentrated reactive and control ACM were performed using Lipidex 1000 resin (Perkin Elmer, 6008301) in disposable columns (Thermo, 29922) according to the manufacturer's protocol. Unbound media was assessed by Bradford Assay to ensure final relative protein concentrations were identical between control and reactive ACM. Bound lipids were eluted with methanol and dried under an argon stream followed by resuspension in methanol to an identical final volume for treatment of cells (with methanol never added to more than 5% final media volume for live dead analysis).

The toxicity of saturated free fatty acids was tested by adding recombinant lipids to oligodendrocyte differentiation media according to Piccolis et al., “Probing the Global Cellular Responses to Lipotoxicity Caused by Saturated Fatty Acids,” Mol. Cell. 74(1):32-44 (2019), which is hereby incorporated by reference in its entirety). FFAs used in this study included fluorescently labeled palmitic acid for visualization (Avanti 810105), and palmitic acid (Avanti 900400) and stearic acid (Avanti 810612) for toxicity studies. The toxicity of saturated phosphatidylcholines was tested by exposing oligodendrocytes to 20:0 PC (Avanti, 850368) in DMSO to circumvent caveats associated with lipoparticle loading and presentation. Live/dead analysis was performed 24 hours later for both FFA and PC studies.

### Cell Membrane Isolation

Cellular subfractionation was achieved by ultracentrifugation. The membrane fraction of this protocol was dried under an argon stream and resuspended in identical volumes of methanol for presentation to cells. Treatment of cells with this extract was denoted as membrane extract and refers to the percentage of total membrane extract added to cells (with methanol never added to more than 5% final media volume for live/dead analysis).

### Reconstituted Lipoparticles

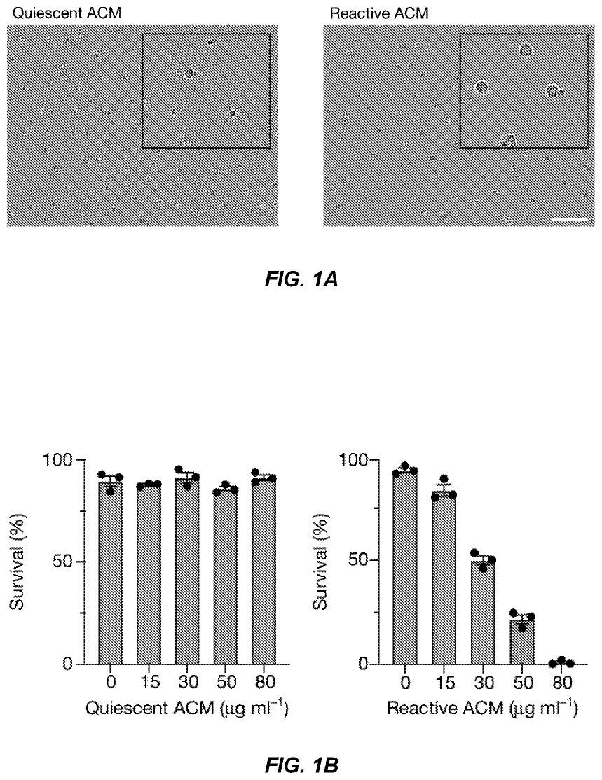

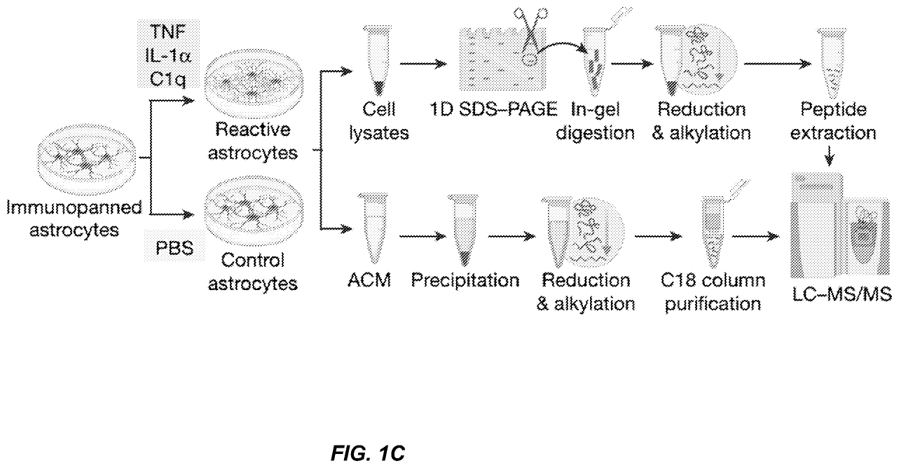

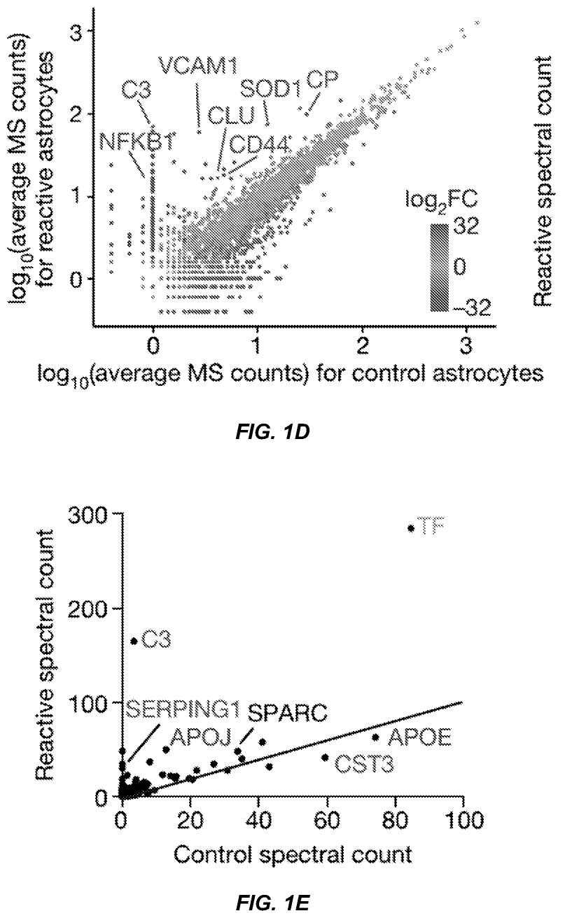

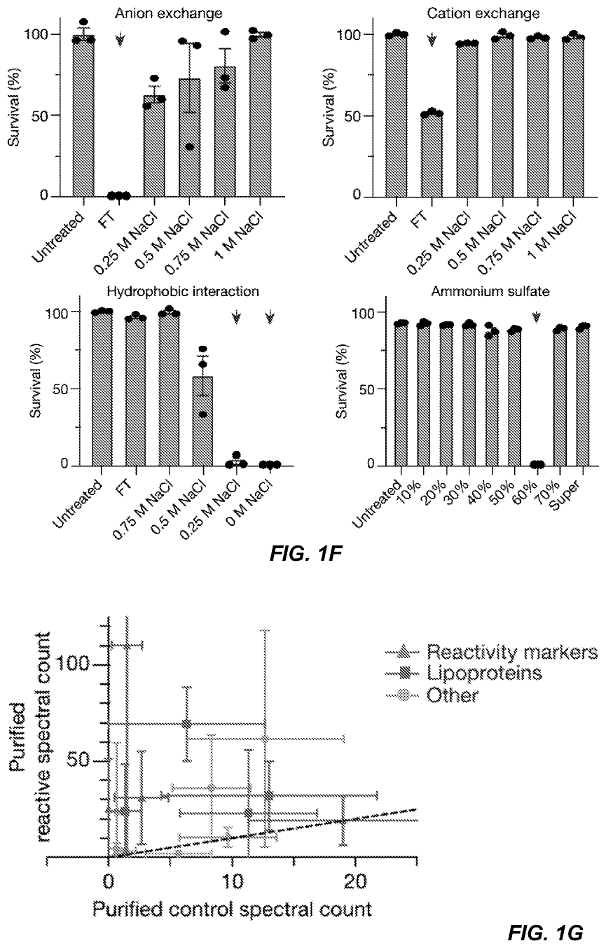

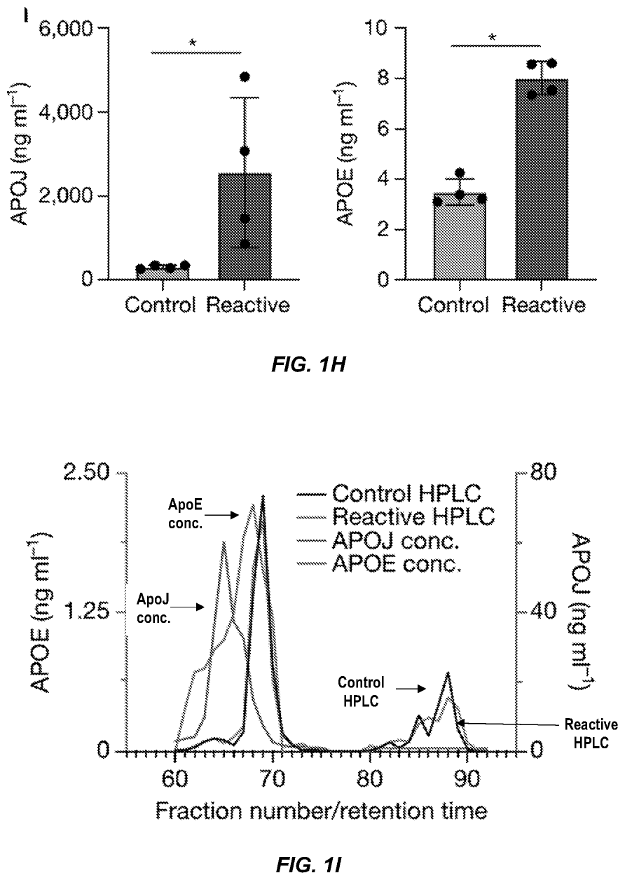

Reconstituted lipoparticles were prepared according to Sparks et al., “The Conformation of Apolipoprotein A-I in Discoidal and Spherical Recombinant High Density Lipoprotein Particles ⁵C NMR Studies of Lysine Ionization Behavior,” J. Biol. Chem. 267(36):25830-258388 (1992), which is hereby incorporated by reference in its entirety. Briefly, desired lipids were added to a 15 ml glass conical tube and dried under an argon stream. Lipids were acquired from identical volumes of identically concentrated ACM by Folch extraction and were spiked with ˜25% TopFluor® PC for visualization (Avanti, 810281). Tris saline (0.01 M Tris, 0.15 M NaCl), pH 8, was then added to give a 20 mM final lipid concentration and the mixture thoroughly vortexed. Sodium cholate in Tris saline was added to a molar ratio of 0.74 lipid/cholate and the mixture vortexed for a further 3 minutes. The dispersion was then incubated at 37° C. and vortexed every 10 minutes until completely clear, usually ˜1 hour. After clearing, the desired amount of ApoE (Fisher, 10817H30E250) and/or ApoJ (Biolegend, 750706) was added and the mixture was diluted to 1 mg protein/ml with Tris buffer and incubated for 1 hour at 37° C. Sodium cholate was removed via extensive dialization against PBS, pH 7.5 and the final preparation was filtered through a 0.22 μm filter. Data in FIG. 1G obtained by treating cells with increasing doses of reconstituted lipoparticles bearing reactive ACM lipids until a minimum dose was found that induces oligodendrocyte cell death and survival then compared to oligodendrocytes treated with an identical volume of identically prepared control-lipid-bearing reconstituted lipoparticles.

### Western Blotting

Protein samples were collected in RIPA buffer (Thermo, 89900) with 1× protease/phosphatase inhibitor (CST, 5872S). The total protein concentration of samples was determined by Bradford assay (Sigma B6916) and equal amounts of total protein were loaded onto 12% Tris-HCl gels (Bio-Rad). Following electrophoresis (100 V for 45 minutes), proteins were transferred to Immobilon-P membranes (EMD Millipore). Blots were probed overnight at 4° C. with 1:1000 GAPDH (ProSci, 3781), 1:500 cleaved caspase 3 (CST, 9661S), 1:500 phospho-PERK (CST, 3179S), 1:500 PERK (CST, 3192S), 1:500 EIF2a (CST, 5324T), 1:500 phospho-Eif2a (CST, 3398T), 1:500 Foxo3a (CST, 12829S), 1:500 phospho-Foxo3a Ser 294 (CST, 5538S), 1:500 Trib3 (Thermo, PA529887), 1:500 ATF3 (Abcam, ab207434), 1:500 CHOP (CST, 5554S), or 1:50 PUMA (Thermo, MA5-31994). Blots were incubated with HRP-conjugated secondary antibodies at 1:5,000 for 2 hours at room temperature and developed using ECL Prime Western Blotting Detection Reagent (GE Healthcare). Visualization and imaging of blots was performed using a Konica Minolta SRX-101a with CL-XPosure Film (Thermo, 34090).

siRNA

siRNAs against rat transcripts were acquired from Dharmacon and included: ON-TARGETplus Non-targeting Control Pool, ON-TARGETplus SMARTpool Scd siRNA, and ON-TARGETplus SMARTpool Insig1 siRNA. siRNAs were transfected into cultured rat OPCs using the basic glial cells nucleofector kit (Lonza) using a Nucleofector 2b Device (Lonza) according to manufacturer's protocol. In brief, 2 million OPCs were resuspended in 100 μl nucleofector solution and electroporated with 15 μl of 20 μM siRNA. Immediately after transfection, cells were diluted in 10 ml DMEM and centrifuged at 250× g for 5 minutes to remove dead cells. Cells were then resuspended in oligodendrocyte proliferation media and a full media change to differentiation media performed the following day. Experiments were performed on mature oligodendrocytes 3 days after transfer to differentiation media as in other experiments. Efficiency of siRNA knockdown validated using qPCR as outlined in the following methods section.

### RNAscope In Situ Hybridization

Fresh frozen mouse eyes were embedded in embedding medium (O.C.T., Sakura), cryosectioned to 20 μm and mounted on Superfrost™ Plus Microscope Slides (Fisher). Fluorescent Multiplex RNAScope (ACD) was performed according to the manufacturer's instructions. Tissue sections were fixed in methanol (15 minutes, 4° C.), sequentially dehydrated in ethanol (50%, 70% and 100% at RT) and enzymatically permeabilized (30 minutes, 40° C., ACD). Tissue was incubated in primary and amplification probes (2 hours primary probe, 30 minutes AMP1, 15 minutes AMP2, 30 minutes AMP3, and 15 minutes AMP4-B at 40° C.) and washed in between steps with RNAScope washing buffer (ACD). Tissue was counterstained with DAPI. After mounting in Fluoromount-G (SothernBiotech), images were acquired on a Keyence BZ-X710 fluorescent microscope using a 20× objective. RNAScope probes were as follows: GFP (Ref.: 409011), Mm-Slc1a3-C3 (Ref.: 430781-C3).

### RNA Extraction, RT-PCR, and Gel Electrophoresis

Following euthanization of mice by inhaled CO₂ and decapitation eyeballs, optic nerves, and brains were immediately dissected and fresh-frozen in OCT compound and stored at −80° C. (as per RNAScope). For RNA extraction, selected samples were released from OCT in ice-cold PBS, the retinae dissected from eyeballs, and retinae and optic nerve digested using QiaShredder columns before RNA extraction using the RNeasy Mini kit and gDNA columns (Qiagen) according to manufacturer's instructions, with on-column DNase treatment (final elution volume: 30 μl). For in vitro experiments, RNA was collected directly from cultures wells using the RNeasy mini kit. RNA quality and integrity was evaluated using an Agilent RNA 6000 Pico assay (Agilent 2100 Bioanalyzer) and only samples with RIN >9.0 were used for downstream analysis.

Reverse transcription was performed using qScript™ cDNA SuperMix (QantaBio) according to the manufacturer's protocol. RT-PCR was performed using GoTaq® Green Master Mix (Promega) using the following primer sequences:

Elovl1,
Fwd-
(SEQ ID NO: 1)
GAAGCACTTCGGATGGTTCG;
Rev-
(SEQ ID NO: 2)
CACCACCAACTCCAGGGAAG;
Gfap,
Fwd-
(SEQ ID NO: 3)
AGAAAGGTTGAATCGCTGGA;
Rev-
(SEQ ID NO: 4)
CGGCGATAGTCGTTAGCTTC;
Rplp0 Fwd:
(SEQ ID NO: 5)
CCTAGAGGGTGTCCGCAATG;
Rplp0 REV:
(SEQ ID NO: 6)
TTGGTGTGAGGGGCTTAGTC;
Scd1 FWD:
(SEQ ID NO: 7)
CCCAAGCTGGAGTACGTCTG;
Scd1 REV:
(SEQ ID NO: 8)
AAATATCCCCCAGAGCAAGGTG;
In
sig1 FWD:
(SEQ ID NO: 9)
GCGTCTACCAGTACACGTCC;
and
(SEQ ID NO: 10)
Insigl REV: ATTCTGCTTCCGCCCTGAAT.

Primers were designed using NCBI primer BLAST software (http://www.ncbi.nlm.nih .gov/tools/primer-blast/) and primer pairs with least probability of amplifying non-specific products as predicted by NCBI primer BLAST were selected. All primers had 90-105% efficiency. Primer pairs were designed to amplify products that spanned exon-exon junctions to avoid amplification of genomic DNA. The specificity of the primer pairs was tested by PCR with mouse whole-brain cDNA (prepared fresh) and PCR products were examined by agarose gel electrophoresis.

For gel electrophoresis, cycling parameters were as follows: 2:00 at 95° C., followed by 30 (Elovl1) or 40 (Gfap) cycles of 95° C. for 1:00, 60° C. for 1:00, 72° C. for 1:00. After cycles a final 5:00 incubation at 72° C. was completed before storage of samples at 4° C. Resultant samples separated on a 1.5% agarose gel run at 100V for 40 minutes. Gel images were taken with Gel Doc™ XR+Imaging System (BioRad) using ImageLab™ Software (version 6.0.0 build 25; BioRad) and Elovl1 bands normalized to Gfap expression in the same samples using the [Analyze>Gels] function in FIJI.

Quantitative RT-PCR was performed using Fast SYBR Green (Applied Biosystems) with a cycling program of 95° C. for 20 seconds followed by 40 cycles of 95° C. for 3 seconds and 60° C. for 30 seconds and ending with a melting curve. Relative mRNA expression was normalized to Rplp0.

### Optic Nerve Crush

P30-P50 mice were anaesthetized with 3.0% inhaled isoflurane in 1.51 O₂ per min. The supero-external orbital contents were blunt-dissected, the superior and lateral rectis muscles teased apart, and the left optic nerve exposed, avoiding any incision to the orbital rim. The nerve was crushed for 3-5 s at approximately 2 mm distal to the lamina cribrosa. After surgery, retinal blood flow was validated by checking the eye fundi. Retinas were collected 14 days after crush and flat mounted for staining with 1:500 guinea pig anti-RBPMS (PhosphoSolutions, 1832-RBPMS) and visualization with 1:1000 Alexa 488 goat anti-guinea pig secondary (Abcam, ab150185). Retinas were imaged on a Zeiss LSM710 Confocal Microscope using Zen 2012 v. 14.09.201 software. Three regions of interest were selected randomly throughout the retina (ensuring multiple eccentricities selected for each retina) blind to genotype and condition and the average number of RBPMS+cells in the three images calculated. One ONC was performed on each animal and the retina of the uncrushed eye used as a within-animal control.

### Data Availability

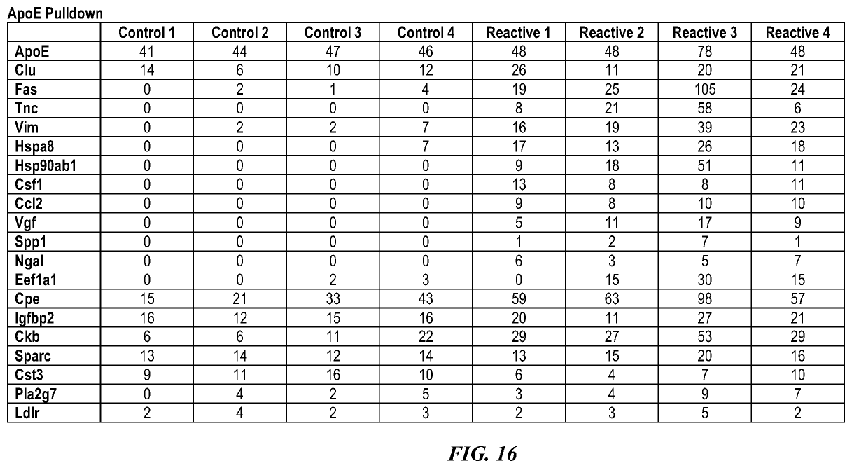

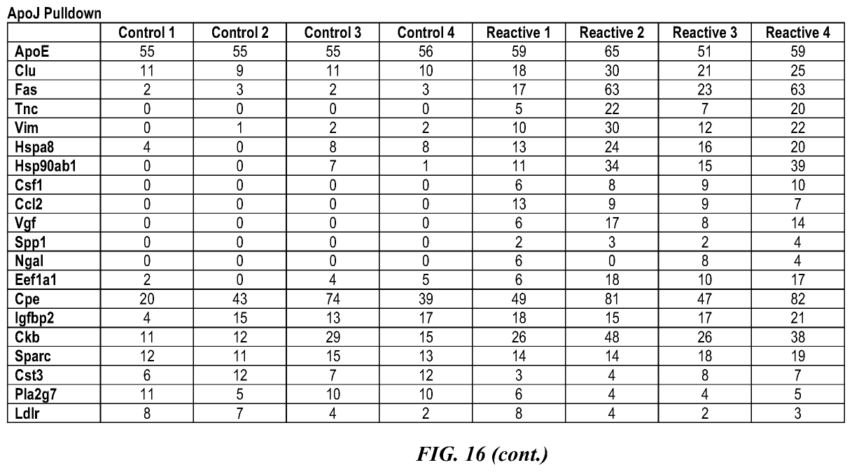

Mass spectrometry data in FIGS. 1, 2, and 4 is available as raw data in FIG. 16 and Guttenplan et al., “Neurotoxic Reactive Astrocyte Induce Cell Death Via Saturated Lipids,” Nature 599:102-107 (2021), which is hereby incorporated by reference in its entirety. Accession information for raw protein mass spectrometry data is MassIVE MSV000087805. Accession information for raw lipid and metabolite mass spectrometry data is MassIVE MSV000087832.

### Code Availability

All statistics were performed using Prism v 8.2.1. Select illustrations in figure subpanels were made using BioRender.

### Example 1—Screening of Reactive Astrocytes for Toxic Proteins

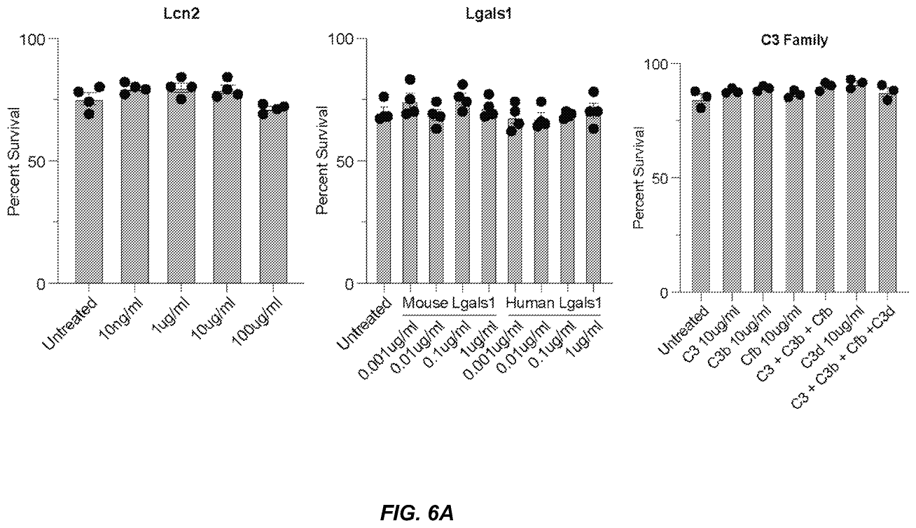

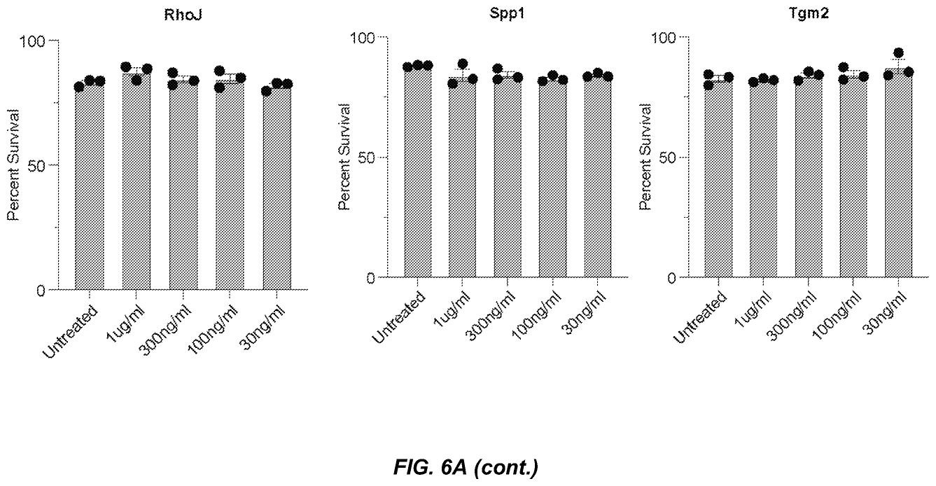

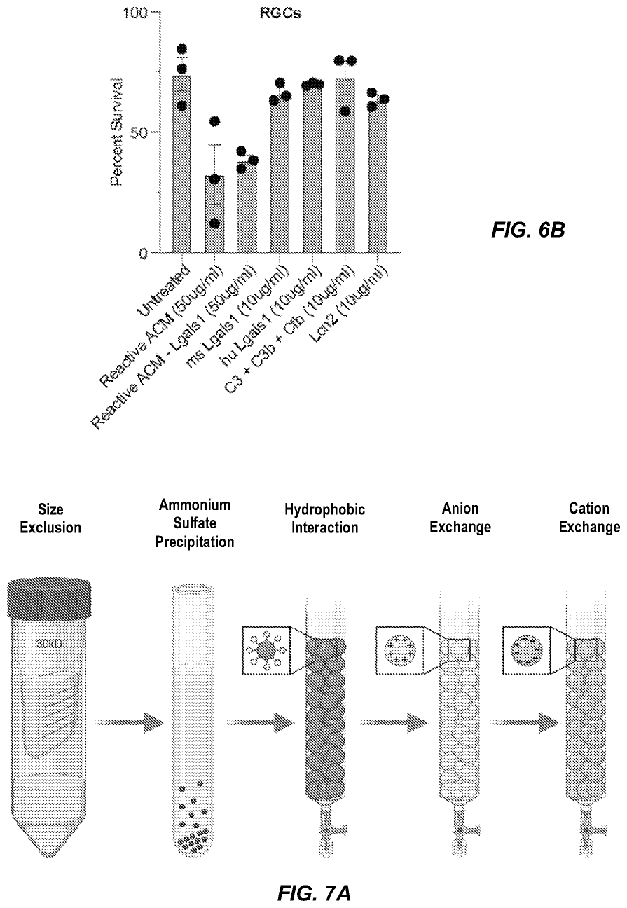

Since previous evidence suggested the toxic activity of reactive astrocytes is mediated by a secreted protein (Nagai et al., “Astrocytes Expressing ALS-Linked Mutated SOD1 Release Factors Selectively Toxic to Motor Neurons,” Nat. Neurosci. 10(5):615-622 (2007) and Giorgio et al., “Non-Cell Autonomous Effect of Glia on Motor Neurons in an Embryonic Stem Cell-Based ALS Model,” Nat. Neurosci. 10(5):608-614 (2007), which are hereby incorporated by reference in their entirety), the identity of the toxic agent was first sought via protein mass spectrometry of reactive versus control astrocyte conditioned media (ACM). Mature oligodendrocytes were used to screen for ACM toxicity, because they are highly viable in serum-free conditions and do not require astrocyte trophic support (FIGS. 1A-1B) (Barres et al., “Multiple Extracellular Signals are Required for Long-Term Oligodendrocyte Survival,” Development 118(1):283-895 (1993), which is hereby incorporated by reference in its entirety), allowing the separation of active toxicity from a lack of trophic support (Banker and Cowan, “Rat Hippocampal Neurons in Dispersed Cell Culture,” Brain Res. 126(3):397-442 (1977), which is hereby incorporated by reference in its entirety). Mass spectrometry was performed on quiescent and reactive astrocytes and their ACM to identify changes in protein abundance following activation (FIGS. 1C-1E). While 176 of 3,660 detected proteins changed in abundance within reactive astrocytes (FIGS. 5A-5D), most of these changes correspond to known reactivity signature genes, with the inflammatory astrocyte protein complement component C3 showing the largest increase (FIG. 5D) (Liddelow et al., “Neurotoxic Reactive Astrocytes are Induced by Activated Microglia,” Nature 541(7638):481-487 (2017), which is hereby incorporated by reference in its entirety). Astrocyte secreted proteins were next considered and, in addition to previously described factors such as SPARC (Kucukdereli et al., “Control of Excitatory CNS Synaptogenesis by Astrocyte-Secreted Proteins Hevin and SPARC,” PNAS 108(32):E440—E449 (2011), which is hereby incorporated by reference in its entirety), the expected increase in abundance of C3, lipocalin-2, and other reactivity markers in reactive ACM was observed (FIG. 1E) (Bi et al., “Reactive Astrocytes Secrete lcn2 to Promote Neuron Death,” P. Natl. Acad. Sci. USA 110(10):4069-4074 (2013), which is hereby incorporated by reference in its entirety). Contrary to prior suggestions in the literature, none of these commonly upregulated reactivity markers drive oligodendrocyte or neuronal death in serum-free in vitro cultures (FIGS. 6A-6B). However, a surprising abundance of lipoparticle proteins (lipoproteins) ApoE and ApoJ in reactive ACM were also noted, indicating a potential change in astrocytic lipid efflux (FIG. 1E).

### Example 2—Identification of Lipoparticle Toxic Agents

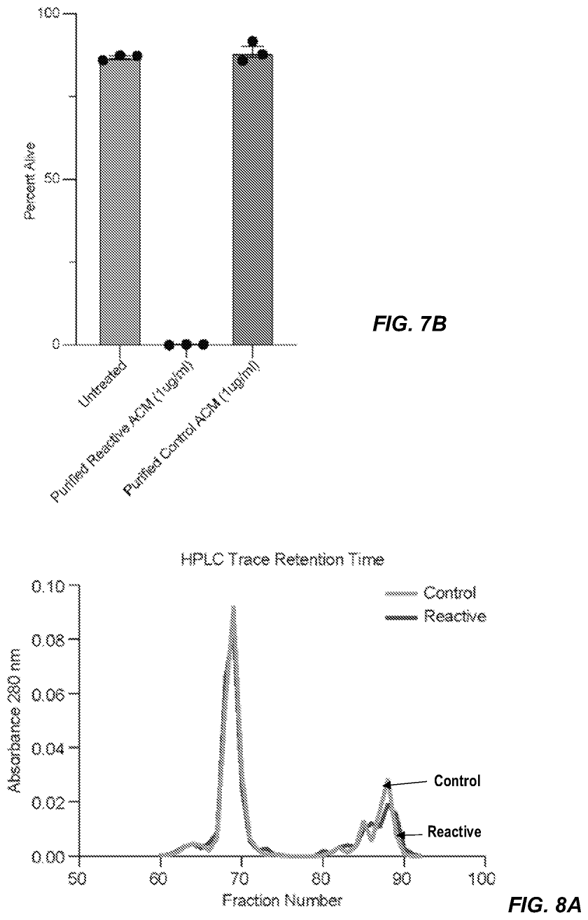

Since proteins that clearly mediate astrocyte toxicity were not detected, reactive ACM was further purified to enrich for toxic activity. Biochemical purification columns were used to separate media by size, charge, and hydrophobicity and fractions were tested for toxicity (FIG. 1F). Toxicity was most prominent in the flow-through of anion and cation exchange columns as well as the final elutions of a hydrophobic interaction chromatography column (FIG. 1F). Next, protein mass spectrometry was performed on control and reactive ACM purified using these columns in series (FIGS. 7A-7B). The most prominent class of proteins upregulated in purified reactive media, other than known markers such as C3, were lipoparticle proteins such as ApoE and ApoJ, again indicating a possible change in astrocyte lipid efflux and highlighting astrocyte-secreted lipoparticles as potential bearers of toxicity (FIG. 1G and Guttenplan et al., “Neurotoxic Reactive Astrocyte Induce Cell Death Via Saturated Lipids,” Nature 599:102-107 (2021), which is hereby incorporated by reference in its entirety).

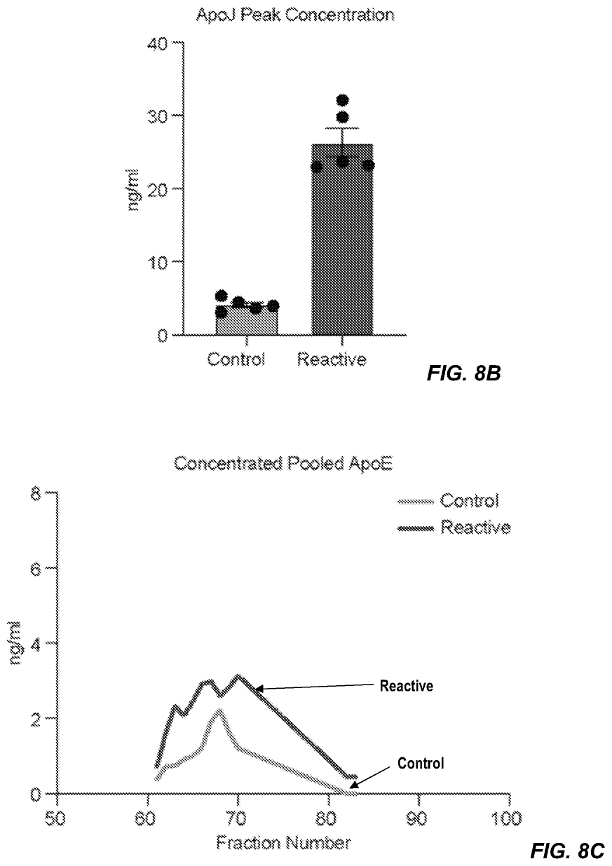

To validate this increase in lipoparticle secretion, ELISAs (enzyme-linked immunosorbent assays) were performed to quantify ApoE and ApoJ protein concentration in control versus reactive ACM. Both lipoproteins were found to be enriched in reactive conditioned media (FIG. 1H). Next, size-exclusion high performance liquid chromatography (HPLC) was performed to identify fractions associated with astrocytic high density lipoparticle (HDL)-like lipoparticles (FIG. 1I, FIGS. 8A-8C). Consistent with the hypothesis that ApoE and ApoJ are predominantly secreted in lipidated lipoparticles, ApoE and ApoJ were found in HDL-like fractions, with an increase in ApoJ concentration within HDL-like fractions (FIGS. 8A-8C) (Fagan et al., “Unique Lipoproteins Secreted by Primary Astrocytes from Wild Type, apoE (-/-), and Human apoE Transgenic Mice,” J. Biol. Chem. 274(42):30001-30007 (1999), which is hereby incorporated by reference in its entirety).

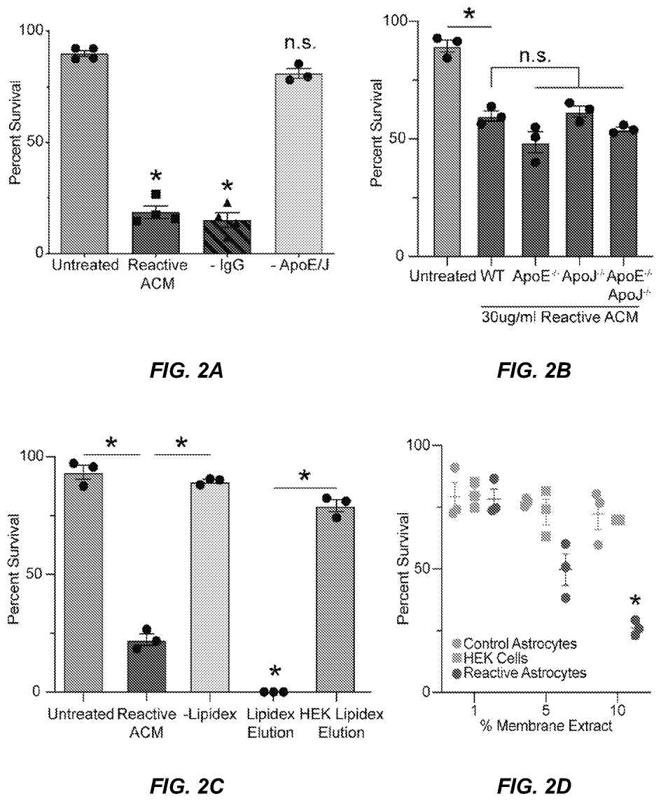

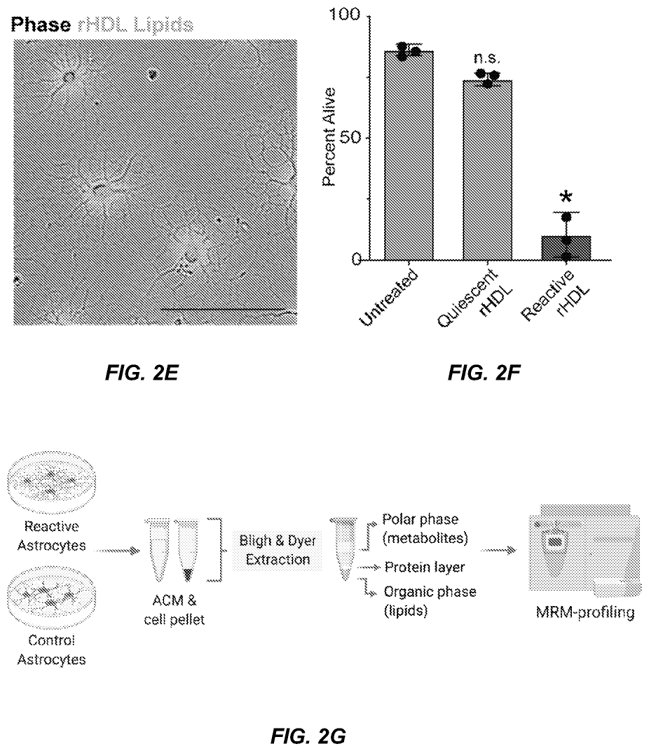

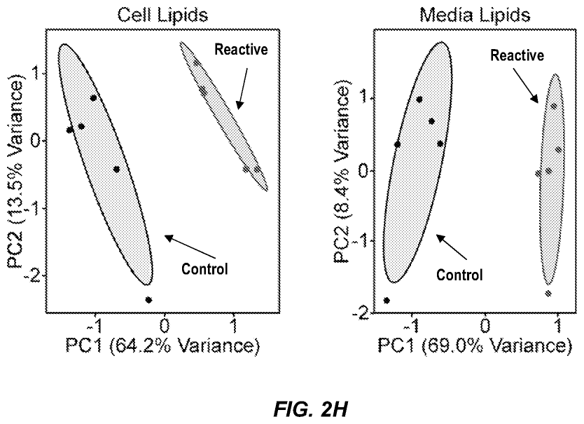

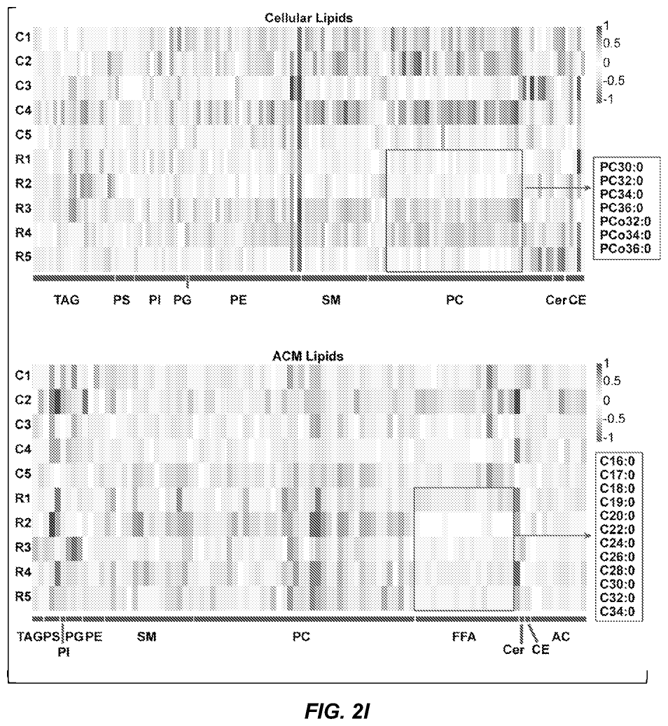

Given the abundance of lipoproteins in purified toxic ACM, as well as the increase of ApoE and ApoJ within reactive astrocyte lipoparticles whether lipoparticles harbor the astrocyte-mediated toxic activity was next investigated. ApoE and ApoJ antibodies were used to immuno-deplete lipoparticles from control and reactive ACM (FIG. 2A, FIG. 16). ACM depleted of ApoE and ApoJ, but not with IgG control antibodies, showed decreased toxicity, indicating that ApoE and ApoJ lipoparticles are necessary for astrocyte-mediated toxicity (FIG. 2A). It was next hypothesized that either the lipoproteins or the lipid content of these lipoparticles mediated the toxic activity. Whether ApoE or ApoJ proteins themselves were toxic was tested by collecting ACM of astrocytes cultured from Apoe^{-/-}, Clu^{-/-} (ApoJ^{-/-}), or Apoe^{-/-}Clu^{-/-} mice (FIG. 2B). ACM toxicity was identical between knockouts and C57B1/6 controls, indicating that the lipoproteins themselves are not toxic (FIG. 2B). In contrast, removing lipids from WT reactive ACM using a Lipidex 3000 column eliminated toxicity (FIG. 2C). Moreover, reactive ACM lipids eluted from the Lipidex 3000 column were toxic (FIG. 2C). These results provide evidence that lipids themselves are both necessary and sufficient components of the ACM toxicity (FIG. 2C). Importantly, saturating quantities of lipids from HEK cell conditioned media were not toxic, indicating that the observed toxicity is not due to a surfactant effect (FIG. 2C). To determine if the toxic lipids are trafficked through the reactive astrocyte membrane, cell lysates were fractionated by ultracentrifugation and oligodendrocytes were treated with lipid extracts of cellular membranes (FIG. 2D). Consistent with a lipid mediator of toxicity, reactive astrocyte membrane fractions were more toxic than the membranes of quiescent astrocytes and HEK cells (FIG. 2D).

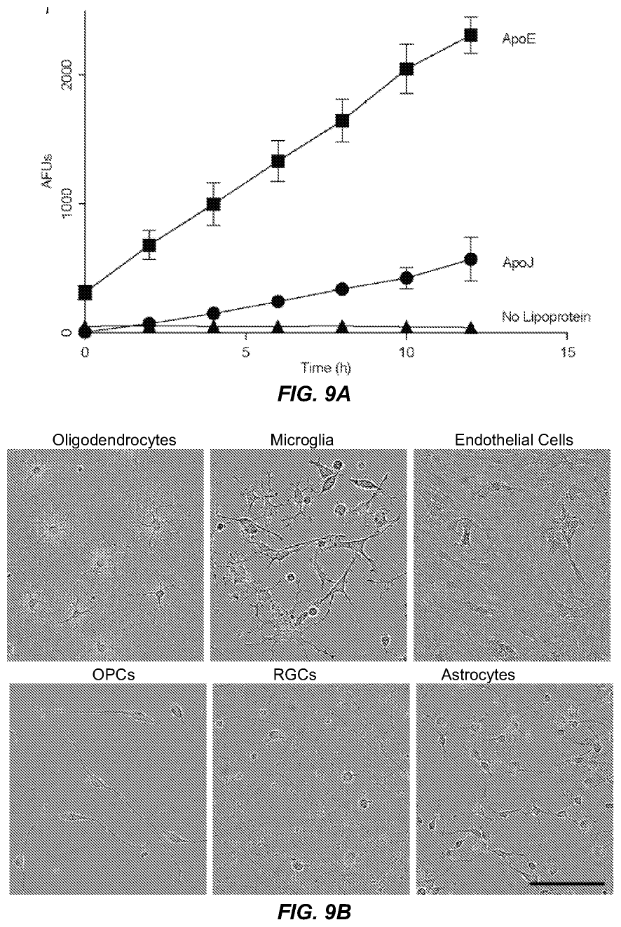

Finally, whether astrocyte-mediated toxicity could be recapitulated using reconstituted lipoparticles containing lipids from reactive ACM was investigated. Folch extractions were performed on control and reactive ACM and these lipids were used to produce ApoE- and ApoJ-containing reconstituted lipoparticles (rHDL) (Folch et al., “A Simple Method for the Isolation and Purification of Total Lipids from Animal Tissues,” J. Biol. Chem. 226(1):497-509 (1957) and Sparks et al., “The Conformation of Apolipoprotein A-I in Discoidal and Spherical Recombinant High Density Lipoprotein Particles 13C NMR Studies of Lysine Ionization Behavior,” J. Biol. Chem. 267(36):25830-258388 (1992), which are hereby incorporated by reference in their entirety). Fluorescently labeled lipids were next incorporated into treated oligodendrocytes to visualize rHDL uptake and, consistent with normal lipoparticle properties, uptake was dependent on cell type and on the presence of lipoparticle proteins (FIG. 2E, FIGS. 9A-9B). Treating oligodendrocytes with quiescent ACM-lipid-containing rHDL was non-toxic, but treatment with reactive ACM-lipid-containing rHDL was highly toxic, providing evidence that toxic lipids were present at higher concentrations in the ACM of reactive astrocytes (FIG. 2F).

### Example 3—Evaluation of Lipoparticle Lipid Components for Toxicity

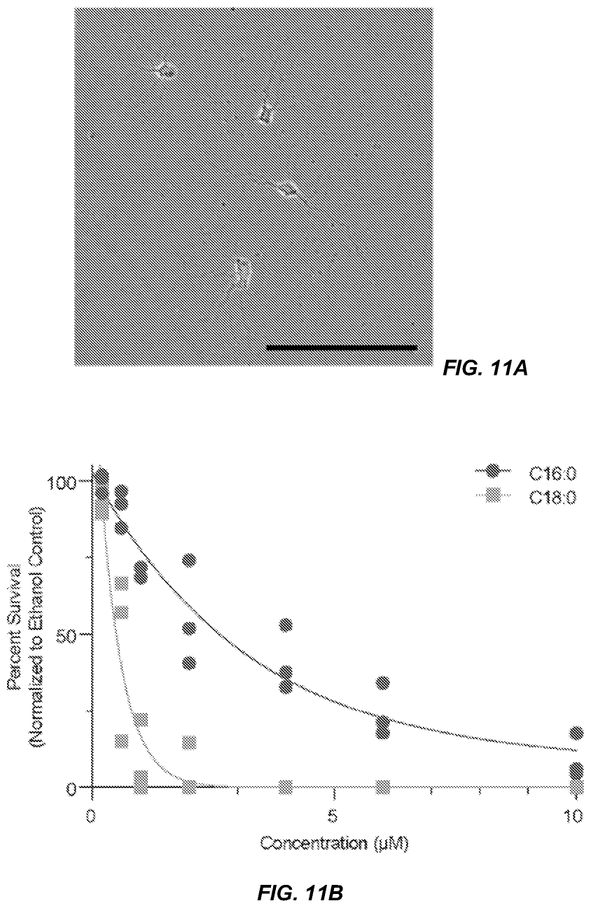

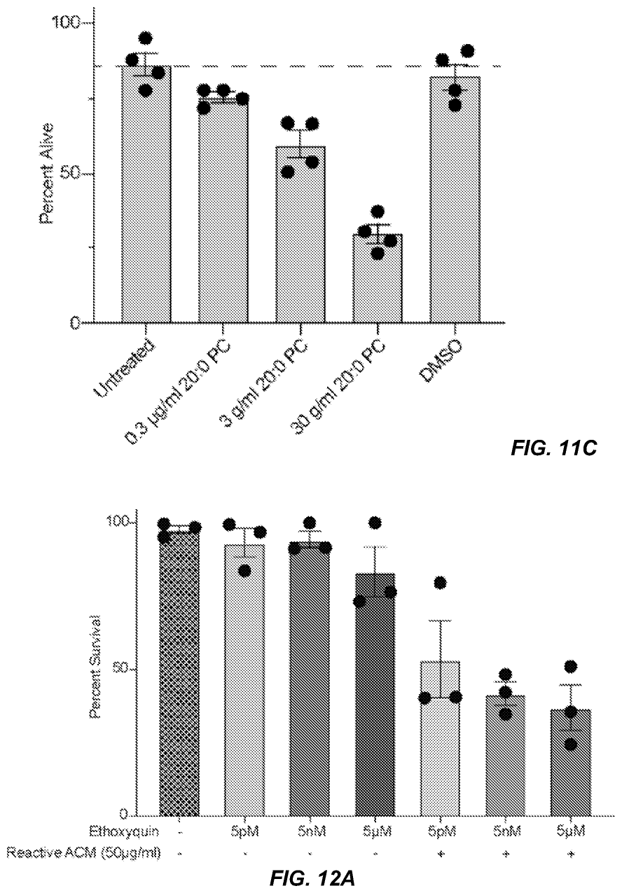

What lipid components of the astrocyte-derived lipoparticles are upregulated in reactive astrocyte conditioned media and could mediate toxicity was next investigated. Unbiased lipidomics and metabolomics (1,501 lipids from 10 classes and 717 metabolites) was performed on cell extracts and ACM from quiescent and reactive astrocytes to determine if there was a shift in the lipidome or metabolome (FIG. 2G). Significant changes in lipid metabolism and, to a lesser extent, the metabolome in reactive compared to quiescent astrocytes was observed (FIG. 2H, FIGS. 10A-10C). Notably, there was a substantial upregulation of phosphatidylcholines with very-long chain fatty acid acyl chains (VLCPCs) in astrocyte cell membranes and long-chain saturated free fatty acids (FFAs) in reactive astrocyte ACM, lipids that are normally of relatively low abundance in membranes and ApoE- and ApoJ-containing lipoparticles (DeMattos et al., “Purification and Characterization of Astrocyte-Secreted Apolipoprotein E and J-Containing Lipoproteins from Wild-Type and Human apoE Transgenic Mice,” Neurochem. Int. 39(5-6):415-425 (2001), which is hereby incorporated by reference in its entirety) (FIG. 2I, FIG. 10D). Both long-chain, saturated FFAs and VLCPCs were toxic to cultured oligodendrocytes, with greater toxicity caused by longer chain lengths (FIGS. 11A-11C). Given the toxicity of both reactive astrocyte membranes (FIG. 2D) and ACM—and the similar mechanisms of cell death caused by long-chain saturated FFAs and phosphatidylcholines—it was concluded that saturated, long-chain FFAs and VLCPCs likely mediate toxicity.

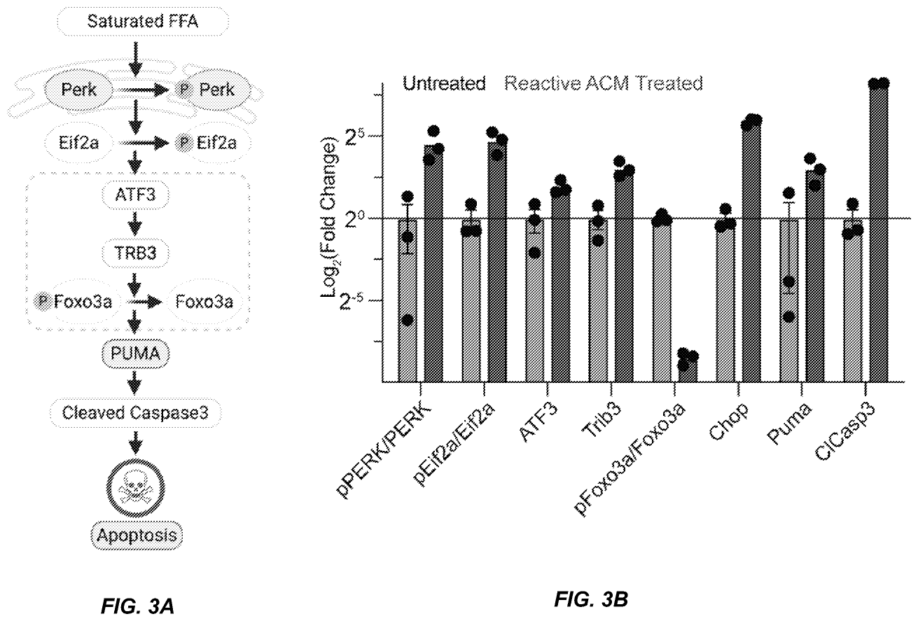

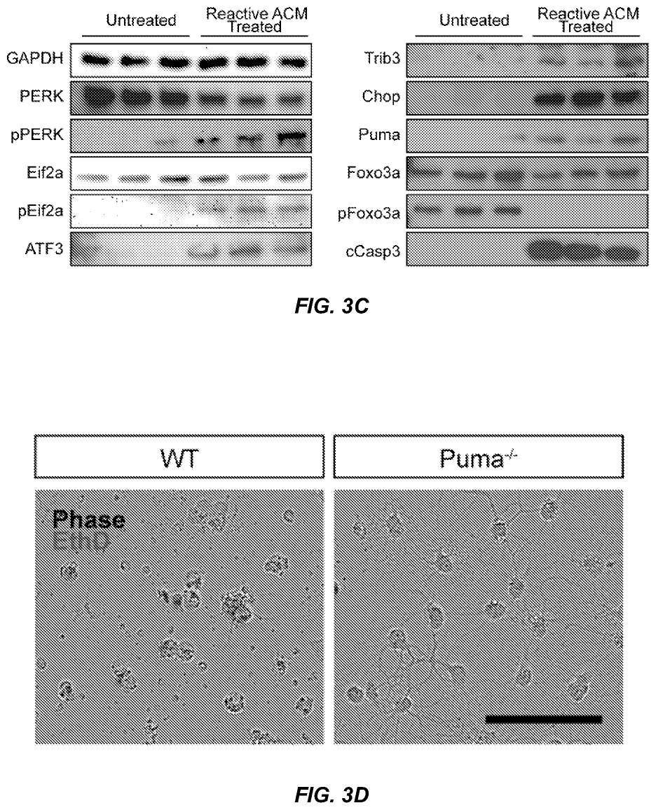

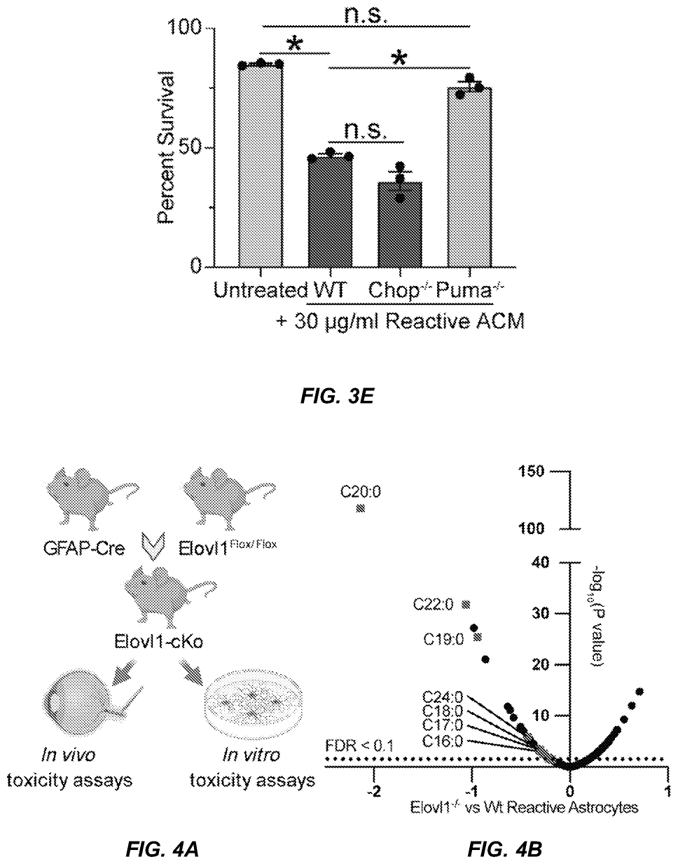

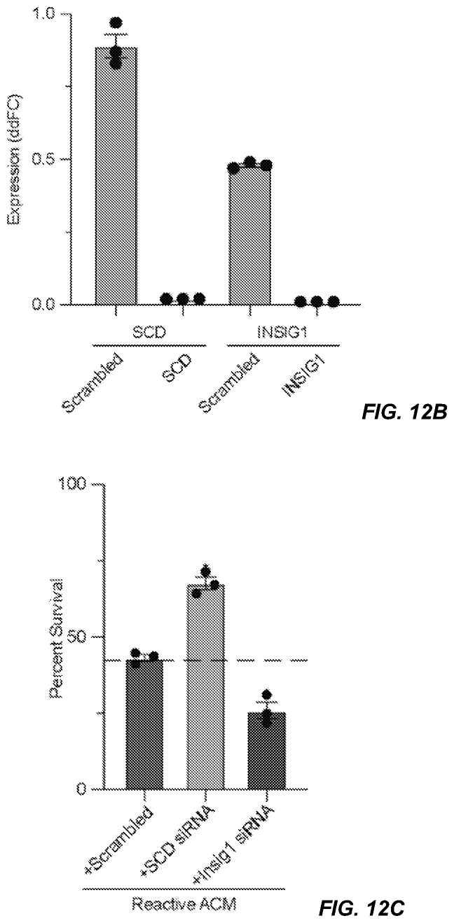

Whether long-chain saturated lipids mediate toxicity, and align with cell death initiated by reactive ACM, was evaluated next. Previous data showed that Ferrostatin-1, an inhibitor of lipid peroxidation, does not affect reactive ACM-mediated toxicity (Liddelow et al., “Neurotoxic Reactive Astrocytes are Induced by Activated Microglia,” Nature 541(7638):481-487 (2017), which is hereby incorporated by reference in its entirety). Further, inhibition of lipid peroxidation by adding the antioxidant ethoxyquin to oligodendrocyte cultures had no effect on astrocyte-driven toxicity (FIG. 12A). These results highlight lipoapoptosis, rather than ferroptosis, as a possible mechanism through which saturated lipids like FFAs and VLCPCs mediate their cytotoxicity. In lipoapoptosis, saturated lipids activate the PERK (Protein Kinase R-like ER Kinase) endoplasmic reticulum stress response pathway likely due to changes in lipid membrane structure, leading to cell death via PUMA (p53 upregulated modulator of apoptosis, Bbc3) or CHOP (C/EBP homologous protein, Ddit3) following dephosphorylation of FOXO3a (FIG. 3A) (Cunha et al., “Death Protein 5 and p53-Upregulated Modulator of Apoptosis Mediate the Endoplasmic Reticulum Stress-Mitochondrial Dialog Triggering Lipotoxic Rodent and Human β-Cell Apoptosis,” Diabetes 61(11):2763-2775 (2012), which is hereby incorporated by reference in its entirety). By western blot, each key lipoapoptosis pathway indicator was observed in oligodendrocytes treated with reactive ACM, including FOXO3a dephosphorylation followed by PUMA upregulation and Caspase 3 cleavage (FIGS. 3B-3C). Sensitivity of oligodendrocytes to changes in lipoapoptosis modulating genes Scd1 and Insig1 was also observed (FIGS. 12B-12C) (Piccolis et al., “Probing the Global Cellular Responses to Lipotoxicity Caused by Saturated Fatty Acids,” Mol. Cell. 74(1):32-44 (2019) and Fanning et al., “Lipidomic Analysis of α-Synuclein Neurotoxicity Identifies Stearoyl CoA Desaturase as a Target for Parkinson Treatment,” Mol. Cell 73(5):1001-1014 (2018), which are hereby incorporated by reference in their entirety). If lipoapoptosis is driving cell death, then PUMA or CHOP should be key mediators of the process. Consistent with this hypothesis, oligodendrocytes from PUMA^{-/-} knockout mice (Bbc3^{-/-}), but not CHOP^{-/-} (Ddit3^{-/-}) mice, are resistant to cell death mediated by reactive ACM (FIGS. 3D-3E).

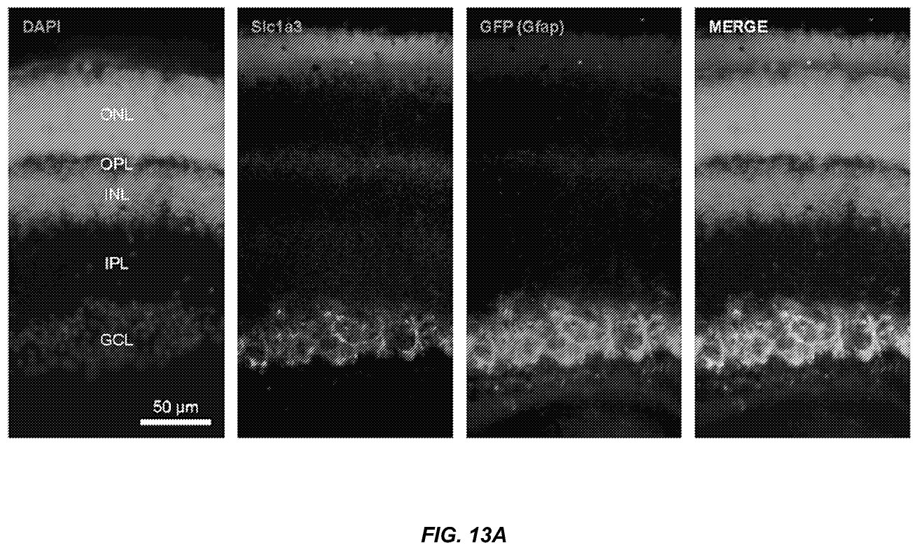

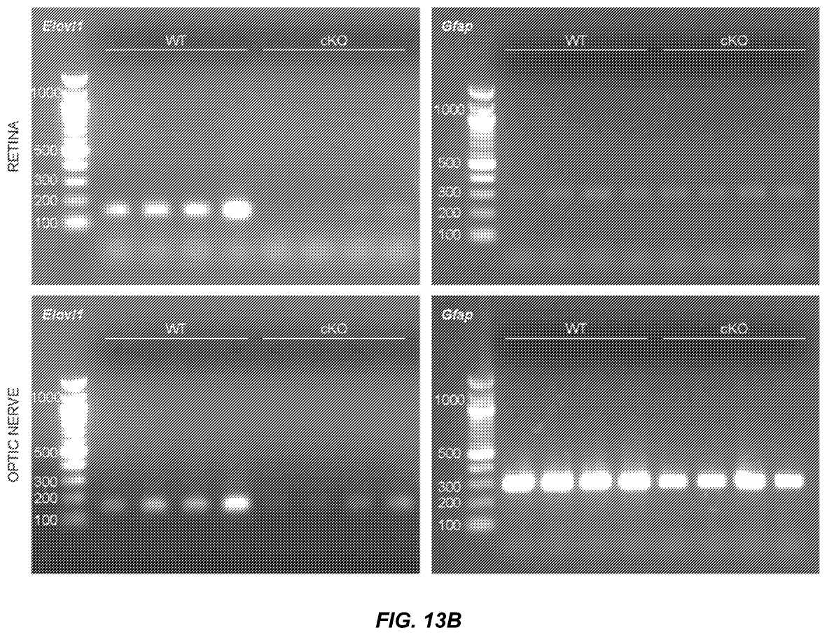

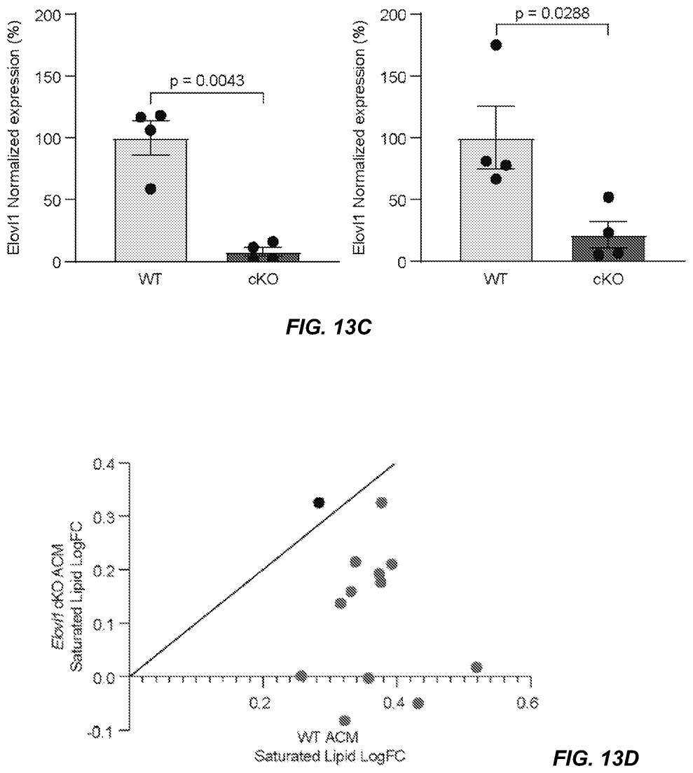

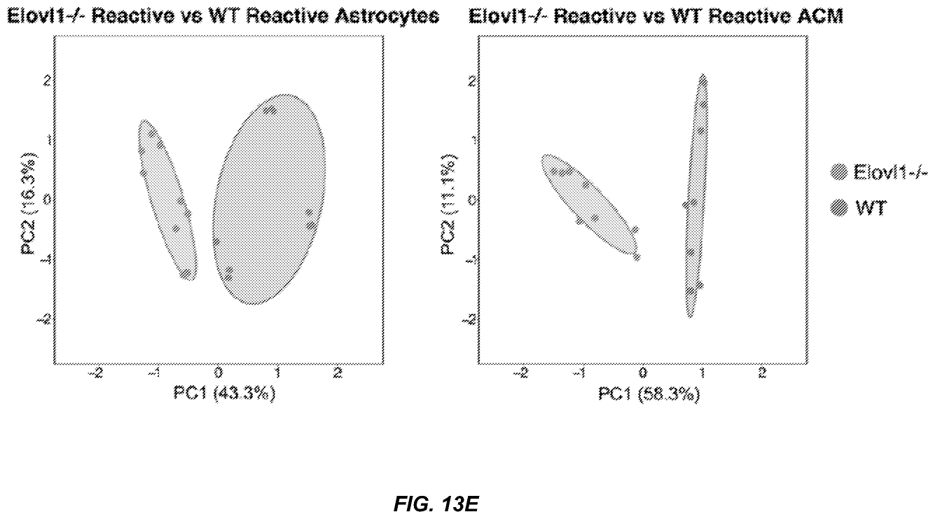

Next, elimination of the production of long-chain saturated lipids was performed to demonstrate their necessity for astrocyte-mediated toxicity. Because shorter length and unsaturated phosphatidylcholines and free fatty acids are essential for cell survival, ELOVL1, the metabolic enzyme specifically responsible for synthesis of longer chain, fully-saturated lipids (≥C16:0) upregulated in reactive astrocytes and ACM (similar enzymes ELOVL3 and ELOVL7 are lowly expressed in astrocytes (Zhang et al., “An RNA-Sequencing Transcriptome and Splicing Database of Glia, Neurons, and Vascular Cells of the Cerebral Cortex,” J. Neurosci. 34(36):11929-11947 (2014), which is hereby incorporated by reference in its entirety) was targeted. An Elovl1^{flox/flox} line was crossed to a Gfap-Cre line to generate an astrocyte-specific Elovl1 conditional knockout mouse (cKO, FIG. 4A, FIGS. 13A-13C). Astrocytes were purified from WT and Elovl1 cKO mice and their lipidomes were compared when quiescent and reactive. As expected, a lower abundance (but not complete elimination) of long-chain saturated FFAs was observed in Elovl1 cKO astrocytes (FIG. 4B, FIGS. 13D-13E). The reactive ACM from Elovl1 cKO mice was significantly less toxic to oligodendrocytes in vitro than the reactive ACM from WT mice (FIGS. 4C-4D, FIG. 14). Concentrating this reactive ACM 10-fold, Elovl1 cKO reactive ACM became toxic to oligodendrocytes, but identically concentrated WT reactive ACM proved much more toxic. Together, these results suggest that saturated lipids mediate the toxic activity of reactive astrocyte and Elovl1 cKO reduces the production of these toxic lipids.

In previous studies, genomic knockout of Il1a, Tnf, and C1qa prevented death of RGCs following retinal injury (Liddelow et al., “Neurotoxic Reactive Astrocytes are Induced by Activated Microglia,” Nature 541(7638):481-487 (2017) and Guttenplan et al., “Neurotoxic Reactive Astrocytes Drive Neuronal Death after Retinal Injury,” Cell Rep. 31(12):107776 (2020), which are hereby incorporated by reference in their entirety). Consistent with astrocytes mediating this RGC toxicity, both reactive ACM and reconstituted lipoparticles bearing reactive ACM lipids are toxic to RGCs in vitro (FIG. 4E), aligning with the known sensitivity of RGCs to lipotoxicity (Yan et al., “Palmitic Acid Triggers Cell Apoptosis in RGC-5 Retinal Ganglion Cells Through the Akt/FoxO1 Signaling Pathway,” Metab. Brain Dis. 32(2):453-460 (2017), which is hereby incorporated by reference in its entirety). To determine if RGC death is mediated by long-chain saturated lipids in vivo, ONCs was performed in Elovl1 cKO versus WT mice. While Elovl1 cKO mice still showed neuronal death following ONC (unlike in Il1a^{-/-}Tnf^{-/-}C1qa^{-/-} mice, which show no RGC death following axon crush (Liddelow et al., “Neurotoxic Reactive Astrocytes are Induced by Activated Microglia,” Nature 541(7638):481-487 (2017); Guttenplan et al., “Neurotoxic Reactive Astrocytes Drive Neuronal Death after Retinal Injury,” Cell Rep. 31(12):107776 (2020); Sterling et al., “GLP-1 Receptor Agonist NLY01 Reduces Retinal Inflammation and Neuron Death Secondary to Ocular Hypertension,” Cell Reports 33(5):108271 (2020), which are hereby incorporated by reference in their entirety), the amount of death was reduced, indicating that long-chain FFAs and PCs partially mediate toxicity of RGCs following axon crush in vivo (FIGS. 4F-4G). These results demonstrate that reactive astrocytes drive neuron cell death by secreting toxic lipids both in vitro and in vivo, identifying at least one long-sought astrocyte-secreted neurotoxic class of molecules.

### Discussion of Examples 1-3

The data presented in the preceding Examples demonstrates that neurotoxic reactive astrocytes drive death of neurons and mature oligodendrocytes via delivery of FFAs and VLCPCs, likely via lipoparticle secretion. These findings highlight the important role of the astrocyte reactivity response in CNS injury and neurodegenerative disease and the relatively unexplored role of lipids in CNS signaling. Much of the scientific focus on astrocytes has been on their secreted proteins, possibly due to technical barriers to studying brain-derived lipids. However, these results demonstrate that astrocytes and microglia secrete and degrade a huge array and quantity of lipids, and further study of lipid-mediated functions, like lipid droplet formation, will likely lead to fruitful discoveries (Ioannou, M. S. et al., “Neuron-Astrocyte Metabolic Coupling Protects against Activity-Induced Fatty Acid Toxicity,” Cell 177(6):1522-1535 (2019), which is hereby incorporated by reference in its entirety). It would also be informative to determine if these lipids are differentially trafficked in reactive astrocytes expressing various ApoE isoforms, as previous research has identified a relationship between lipoprotein isoform and lipidation status (Fagan et al., “Unique Lipoproteins Secreted by Primary Astrocytes from Wild Type, apoE (-/-), and Human apoE Transgenic Mice,” J. Biol. Chem. 274(42):30001— 30007 (1999); DeMattos et al., “Purification and Characterization of Astrocyte-Secreted Apolipoprotein E and J-Containing Lipoproteins from Wild-Type and Human apoE Transgenic Mice,” Neurochem. Int. 39(5-6):415-425 (2001); and Legleiter et al., “In situ AFM Studies of Astrocyte-Secreted Apolipoprotein E-and J-Containing Lipoproteins,” J. Colloid Interface Sci. 278(1):96-106 (2004), which are hereby incorporated by reference in their entirety). Given that lipid trafficking is the primary CNS function of lipoproteins, further study of glial lipid metabolism will yield a better understanding of how these proteins may participate in neurodegeneration and whether they could serve as disease biomarkers.

Other mechanisms have been proposed for how astrocytes may exert their toxic influence on CNS cells. While a substantial portion of the toxicity seen in neuroinflammatory reactive ACM seems to be explained by saturated lipids, it is clear that reducing these lipids does not completely eliminate neurotoxicity. Future work will hopefully discover other astrocyte-derived toxins or show that the remaining toxicity can be explained by the incomplete knockdown of saturated lipids in this study or by the influence of already proposed toxic proteins (Bi et al., “Reactive Astrocytes Secrete lcn2 to Promote Neuron Death,” P. Natl. Acad. Sci. USA 110(10):4069-4074 (2013) and Mishra et al., “Systematic Elucidation of Neuron-Astrocyte Interaction in Models of Amyotrophic Lateral Sclerosis Using Multi-Modal Integrated Bioinformatics Workflow,” Nat. Commun. 11(1):5579 (2020), which are hereby incorporated by reference in their entirety), miRNAs (Jovic̆ić and Gitler, “Distinct Repertoires of microRNAs Present in Mouse Astrocytes Compared to Astrocyte-Secreted Exosomes,” Plos One 12(2):e0171418 (2017), which is hereby incorporated by reference in its entirety), or other lipids (Ioannou, M. S. et al., “Neuron-Astrocyte Metabolic Coupling Protects against Activity-Induced Fatty Acid Toxicity,” Cell 177(6):1522-1535 (2019), which is hereby incorporated by reference in its entirety). Other studies report that astrocyte-secreted factors can have context-dependent functions (Guttenplan et al., “Knockout of Reactive Astrocyte Activating Factors Slows Disease Progression in an ALS Mouse Model,” Nat. Commun. 11(1):3753 (2020), which is hereby incorporated by reference in its entirety). Thus, any proposed toxic or trophic molecules should be considered in the context of interest (e.g., disease associated mutations) before assuming their function or mechanism of cell death.

The presented data do not explain why astrocytes upregulate saturated lipids in response to CNS injury or disease. However, upregulation of this lipid class is a common phenomenon in TLR4-mediated immune cell activation (Oishi et al., “SREBP1 Contributes to Resolution of Pro-inflammatory TLR4 Signaling by Reprogramming Fatty Acid Metabolism,” Cell Metab. 25(2):412-427 (2017), which is hereby incorporated by reference in its entirety). Because a peripheral mechanism to inactivate lipopolysaccharide (LPS) is the loading of its lipid component onto lipoparticles (Wurfel et al., “Lipopolysaccharide (LPS)-Binding Protein is Carried on Lipoproteins and Acts as a Cofactor in the Neutralization of LPS,” J. Exp. Med. 180(3):1025-1035 (1994), which is hereby incorporated by reference in its entirety), the reactive response to systemic LPS administration may mirror this peripheral defense. Similarly, microglia change their lipid metabolism in response to the lipid flux that occurs when neurons and oligodendrocytes die in neurodegenerative contexts (Nugent et al., “TREM2 Regulates Microglial Cholesterol Metabolism upon Chronic Phagocytic Challenge,” Neuron. 105(5):837-854 (2020), which is hereby incorporated by reference in its entirety), indicating that astrocytes may respond to a buildup of lipids that occurs during neurodegeneration.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of inhibiting reactive astrocyte mediated neuronal and/or oligodendrocyte cell death in a subject, said method comprising:
administering an inhibitor of Elongation of Very Long Chain Fatty Acids Protein 1 (ELOVL1) to a subject having or at risk of having a condition mediated by reactive astrocytes, wherein said ELOVL1 inhibitor is administered in an amount effective to inhibit reactive astrocyte mediate neuronal and/or oligodendrocyte cell death in the subject.

2. The method of claim 1, wherein the ELOVL1 inhibitor comprises rapamycin, a derivative, or analog thereof.

3. The method of claim 1, wherein the ELOVL1 inhibitor comprises a fibrate.

4. The method of claim 3, wherein the fibrate is bezafibrate or an ester thereof.

5. The method of claim 3, wherein the fibrate is gemfibrozil or an ester thereof.

6. The method of claim 1, wherein the ELOVL1 inhibitor comprises oleic acid, a derivative or analog thereof.

7. The method of claim 1, wherein the ELOVL1 inhibitor comprises erucic acid, a derivative or analog thereof.

8. The method of claim 1, wherein the ELOVL1 inhibitor comprises a mixture of oleic acid and erucic acid.

9. The method of claim 8, wherein the ELOVL1 inhibitor is a 4:1 mixture of oleic acid and erucic acid (Lorenzo's oil).

10. The method of claim 1, wherein the ELOVL1 inhibitor is an inhibitory nucleic acid molecule selected from an ELOVL1 siRNA, ELOVL1 antisense oligonucleotide, and ELOVL1 microRNA.

11. The method of claim 10, wherein the ELOVL1 inhibitory nucleic acid molecule comprises miR-196a.

12. The method of claim 1, wherein the condition mediated by reactive astrocytes is a neurodegenerative disease.

13. The method of claim 12, wherein the neurodegenerative disease is selected from the group consisting of Alzheimer's disease, Parkinson's disease, Huntington's disease, multiple sclerosis, amyotrophic lateral sclerosis, and prion disease.

14. The method of claim 1, wherein the condition mediated by reactive astrocytes is brain cancer.

15. The method of claim 14, wherein the brain cancer is a metastatic brain cancer.

16. The method of claim 14, wherein the brain cancer is oligodendroglioma.

17. The method of claim 1, wherein the condition mediated by reactive astrocytes is glaucoma.

18. The method of claim 1, wherein the condition mediated by reactive astrocytes is traumatic brain injury or acute axonopathy.

19. The method of claim 1, wherein the condition mediated by reactive astrocytes is diabetes.

20. The method of claim 1, wherein the condition mediated by reactive astrocytes is a leukodystrophy.

21. A method of inhibiting reactive astrocyte mediated neuronal and/or oligodendrocyte cell death in a subject, said method comprising:
administering an inhibitor of lipoapoptosis to a subject having or at risk of having a condition mediated by reactive astrocytes, wherein said inhibitor of lipoapoptosis is administered in an amount effective to inhibit reactive astrocyte mediate neuronal and/or oligodendrocyte cell death in the subject.

22. The method of claim 21, wherein the inhibitor of lipoapotosis is an inhibitor of p53 upregulated modulator of apoptosis (PUMA).

23. The method of claim 22, wherein the PUMA inhibitor is CLZ-8 having the following structure 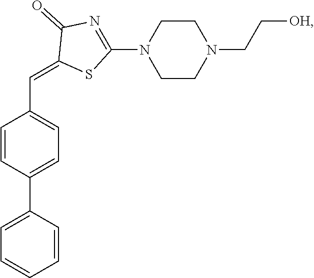 [SMILES: O=C1N=C(N2CCN(CCO)CC2)S/C1=C\c1ccc(-c2ccccc2)cc1]
or an analog or derivative thereof.

24. The method of claim 21, wherein the condition mediated by reactive astrocytes is a neurodegenerative disease.

25. The method of claim 24, wherein the neurodegenerative disease is selected from the group consisting of Alzheimer's disease, Parkinson's disease, Huntington's disease, multiple sclerosis, amyotrophic lateral sclerosis, and prion disease.

26. The method of claim 21, wherein the condition mediated by reactive astrocytes is brain cancer.

27. The method of claim 26, wherein the brain cancer is a metastatic brain cancer.

28. The method of claim 26, wherein the brain cancer is oligodendroglioma.

29. The method of claim 21, wherein the condition mediated by reactive astrocytes is glaucoma.

30. The method of claim 21, wherein the condition mediated by reactive astrocytes is traumatic brain injury or acute axonopathy.

31. The method of claim 21, wherein the condition mediated by reactive astrocytes is diabetes.

32. The method of claim 21, wherein the condition mediated by reactive astrocytes is a leukodystrophy.


SEQUENCE LISTING (10 sequences)

SEQ ID NO: 1 - DNA, 20 bp, Artificial Sequence
  Elovl1, Fwd
gaagcacttcggatggttcg

SEQ ID NO: 2 - DNA, 20 bp, Artificial Sequence
  Elovl1, Rev
caccaccaactccagggaag

SEQ ID NO: 3 - DNA, 20 bp, Artificial Sequence
  Gfap, Fwd
agaaaggttgaatcgctgga

SEQ ID NO: 4 - DNA, 20 bp, Artificial Sequence
  Gfap, Rev
cggcgatagtcgttagcttc

SEQ ID NO: 5 - DNA, 20 bp, Artificial Sequence
  Rplp0 Fwd
cctagagggtgtccgcaatg

SEQ ID NO: 6 - DNA, 20 bp, Artificial Sequence
  Rplp0 REV
ttggtgtgaggggcttagtc

SEQ ID NO: 7 - DNA, 20 bp, Artificial Sequence
  Scd1 FWD
cccaagctggagtacgtctg

SEQ ID NO: 8 - DNA, 22 bp, Artificial Sequence
  Scd1 REV
aaatatcccccagagcaaggtg

SEQ ID NO: 9 - DNA, 20 bp, Artificial Sequence
  Insig1 FWD
gcgtctaccagtacacgtcc

SEQ ID NO: 10 - DNA, 20 bp, Artificial Sequence
  Insig1 REV
attctgcttccgccctgaat